(19) United States
(12) Patent Application Publication
(10) Pub. No.: US 2022/0211868 A1
(43) Pub. Date: Jul. 7, 2022

(54) NANOPARTICLES COMPRISING NON-CLASSICAL MHC AND USES THEREOF

(71) Applicant: UTI Limited Partnership, Calgary (CA)

(72) Inventor: Pedro SANTAMARIA, Calgary (CA)

(21) Appl. No.: 17/532,578

(22) Filed: Nov. 22, 2021

Related U.S. Application Data
(63) Continuation of application No. PCT/US2020/034233, filed on May 22, 2020.
(60) Provisional application No. 62/852,055, filed on May 23, 2019.

(51) Int. Cl. A61K 47/69; A61K 47/54; C07K 14/74; A61K 47/60; A61P 37/06; A61P 1/16; A61P 29/00

(52) U.S. Cl. CPC A61K 47/6929; A61K 47/549; C07K 14/70539; A61P 29/00; A61P 37/06; A61P 1/16; A61K 47/60

(57) ABSTRACT

Described herein are non-classical MHC-nanoparticle complexes that expand invariant NKT cells. Expansion of invariant NKT cells are useful in the treatment of autoimmune or inflammatory disorders

### CROSS-REFERENCE

This application claims the benefit of U.S. Provisional Application 62/852,055 filed on May 23, 2019, which is incorporated by reference herein in its entirety.

Type 1 or invariant NKT (iNKT) cells comprise a subset of T lymphocytes that express an invariant TCR-alpha chain. These lymphocytes recognize exogenous or endogenous glycolipids presented by a non-polymorphic non-classical MHC class I molecule, CD1d. These T-cells are poised to rapidly produce significant amounts of Th1 (IFN-γ)-, Th2 (IL-4 and IL-10)- or Th17 (IL-17)-type cytokines upon activation in a context-dependent manner and have been implicated in the pathogenesis and regulation of a variety of inflammatory disorders. See Bendelac, A., et al. (2007) “The biology of NKT cells.” Annu. Rev. Immunol. 25, 297-336. The lipid alpha-galactosylceramide (αGalCer) is a potent CD1d-restricted ligand for most mouse and human iNKT cells, and has been employed to elicit the adjuvant activity of iNKT cells in vaccination strategies as well as cancer, in part by promoting the iNKT-induced, co-stimulation-dependent maturation of DCs into mature APCs. See Cerundolo, V., et al. (2009) “Harnessing invariant NKT cells in vaccination strategies.” Nat. Rev. Immunol. 9, 28-38. Given the phenotypic and functional heterogeneity of iNKT cells, systemic αGalCer delivery can result in the activation of iNKT cell subsets with opposing functions.

### SUMMARY

The composition and methods described herein can harness and direct immune responses to ceramides/sphingolipids to develop anti-autoimmune and/or anti-inflammatory responses. Described herein are compositions and methods useful for the treatment of autoimmune and inflammatory disorders. The compositions comprise nanoparticles coupled to non-classical MHC molecules. The non-classical MHC molecules comprise a sphingolipid NKT ligand bound to the binding cleft of the non-classical MHC molecule. A complex is formed between the sphingolipid ligand and the non-classical MHC, which is coupled to a nanoparticle (ncMHC-NP). Sphingolipids administered in this context preferentially activate suppressive invariant NKT cells, and thus potentially activate anti inflammatory responses that can combat inflammatory and autoimmune disorders.

A plurality of the non-classical MHC molecule nanoparticles can be included with a suitable pharmaceutically acceptable carrier, excipient, or diluent in a formulation used as medicament for, or in a method of, treating an autoimmune or inflammatory disease. Diseases that afflict organs with large numbers of iNKT cells, such as the liver, may show strong responses to therapeutic intervention with the compositions of nanoparticles coupled to non-classical MHC molecules bound to sphingolipids described herein.

Described herein is a non-classical MHC-nanoparticle comprising: a sphingolipid or an analog thereof; a non-classical MHC molecule; and a nanoparticle; wherein the sphingolipid or the analog thereof is associated with a binding groove of a non-classical MHC molecule, and wherein the non-classical MHC molecule is coupled to the nanoparticle. In certain embodiments, the non-classical MHC-NP does not comprise an immune activating co-stimulatory molecule or cytokine/cytokine receptor. In certain embodiments, the non-classical MHC-nanoparticle comprises a sphingolipid or an analog thereof. In certain embodiments, the sphingolipid or the analog thereof comprises a ceramide or an analog thereof. In certain embodiments, the ceramide or the analog thereof comprises alpha-galactosylceramide (KRN7000), alpha-C-galactosylceramide, alpha-glucuronosyl ceramide, beta-galactosylceramide, PBS-20, PBS-25, sulfatide, isoglobotriosylceramide (iGb3), or combinations thereof. In certain embodiments, the ceramide or an analog thereof comprises alpha-galactosylceramide (KRN7000). In certain embodiments, the non-classical MHC comprises CD1d. In certain embodiments, the CD1d is human CD1d. In certain embodiments, the CD1d comprises an amino acid residue sequence comprising at least about 90%, 95%, 97%, 98%, 99% identity to, or is identical to, any one of SEQ ID NOs: 1, 2, and 3. In certain embodiments, the CD1d comprises an amino acid residue sequence comprising at least about 90%, 95%, 97%, 98%, 99% identity to, or is identical to SEQ ID NO: 3. In certain embodiments, the CD1d comprises an amino acid residue sequence identical to SEQ ID NO: 3. In certain embodiments, the CD1d comprises an amino acid residue sequence comprising at least about 90%, 95%, 97%, 98%, 99% identity to, or is identical to SEQ ID NO: 4. In certain embodiments, the CD1d comprises an amino acid residue sequence identical to SEQ ID NO: 4. In certain embodiments, the non-classical MHC-nanoparticle comprises a β₂ microglobulin. In certain embodiments, the β₂ microglobulin comprises an amino acid residue sequence comprising at least about 90%, 95%, 97%, 98%, 99% identity to, or is identical to SEQ ID NO: 5. In certain embodiments, the β₂ microglobulin comprises an amino acid residue sequence identical to SEQ ID NO: 5. In certain embodiments, the nanoparticle comprises a metal, a metal oxide, a metal sulfide, a metal selenide, or a polymer. In certain embodiments, the metal or metal oxide comprises iron, iron oxide or gold. In certain embodiments, the metal or metal oxide comprises iron or iron oxide. In certain embodiments, the diameter of the nanoparticle is from about 1 nanometer to about 100 nanometers. In certain embodiments, the diameter is from about 5 nanometers to about 25 nanometers. In certain embodiments, the non-classical MHC molecule is covalently coupled to the nanoparticle. In certain embodiments, the non-classical MHC molecule is covalently coupled to the nanoparticle by a polymer linker. In certain embodiments, the polymer comprises dextran. In certain embodiments, the polymer linker is less than about 5 kilodaltons in size. In certain embodiments, the polymer linker comprises polyethylene glycol (PEG). In certain embodiments, the non-classical MHC molecule is coupled to the nanoparticle at a ratio of at least 10:1. In certain embodiments, the non-classical MHC molecule is coupled to the nanoparticle at a ratio of no more than about 1000:1. In certain embodiments, the non-classical MHC molecule is coupled to the nanoparticle at a ratio of no more than about 500:1. In certain embodiments, the non-classical MHC molecule is coupled to the nanoparticle at a ratio of no more than about 100:1. In certain embodiments, described herein, is a composition comprising a plurality of the non-classical MHC-nanoparticles described herein, and a pharmaceutically acceptable excipient, diluent, or carrier. In certain embodiments, the composition is formulated for intravenous administration. In certain embodiments, the composition is formulated for subcutaneous administration. In certain embodiments, the composition comprising non-classical MHC-nanoparticles is for use in treating an autoimmune or inflammatory disorder. In certain embodiments, the autoimmune or inflammatory disorder comprises Type I diabetes, transplantation rejection, multiple sclerosis, multiple-sclerosis related disorder, premature ovarian failure, scleroderma, Sjogren's disease/syndrome, lupus, vitiligo, alopecia (baldness), polyglandular failure, Grave's disease, hypothyroidism, polymyositis, pemphigus, Crohn's disease, colitis, autoimmune hepatitis, hypopituitarism, myocarditis, Addison's disease, autoimmune skin diseases, uveitis, pernicious anemia, hypoparathyroidism, rheumatoid arthritis, asthma, allergic asthma, autoimmune hepatitis, primary sclerosing cholangitis, primary biliary cirrhosis, cirrhosis, neuromyelitis optica spectrum disorder (Devic's disease, opticospinal multiple sclerosis (OSMS)), pemphigus vulgaris, inflammatory bowel disease (IBD), systemic lupus erythematosus (SLE), celiac disease, psoriasis, autoimmune cardiomyopathy, idiopathic dilated cardiomyopathy (IDCM), myasthenia gravis, uveitis, ankylosing spondylitis, Immune Mediated Myopathies (IMM), anti-phospholipid syndrome (ANCA+), atherosclerosis, dermatomyositis, chronic obstructive pulmonary disease (COPD), emphysema, spinal cord injury, ANCA-associated vasculitis, idiopathic pulmonary fibrosis, pulmonary hypertension, non-alcoholic fatty liver disease (NAFLD), non-alcoholic steatohepatitis (NASH) or psoriasis. In certain embodiments, the autoimmune or inflammatory disorder comprises autoimmune hepatitis, primary sclerosing cholangitis, primary biliary cirrhosis, non-alcoholic fatty liver disease (NAFLD), non-alcoholic steatohepatitis (NASH) or cirrhosis. In certain embodiments, the composition comprising non-classical MHC-nanoparticles is for use in creating or expanding immunoregulatory invariant NKT cells. In certain embodiments, the immunoregulatory invariant NKT cells are liver invariant NKT cells. In certain embodiments, the immunoregulatory invariant NKT cells express MAF. In certain embodiments, the immunoregulatory invariant NKT cells express IL-10 and/or IL-21. In certain embodiments, the immunoregulatory invariant NKT cells express any one or more of LAGS, CTLA4, SLAMF6 and ITGA4. Also, described herein, is a method of treating an autoimmune or inflammatory disorder in an individual comprising administering to the individual the non-classical MHC-nanoparticles or a composition of the non-classical MHC-nanoparticles. In certain embodiments, the autoimmune or inflammatory disorder comprises Type I diabetes, transplantation rejection, multiple sclerosis, multiple-sclerosis related disorder, premature ovarian failure, scleroderma, Sjogren's disease/syndrome, lupus, vitiligo, alopecia (baldness), polyglandular failure, Grave's disease, hypothyroidism, polymyositis, pemphigus, Crohn's disease, colitis, autoimmune hepatitis, hypopituitarism, myocarditis, Addison's disease, autoimmune skin diseases, uveitis, pernicious anemia, hypoparathyroidism, rheumatoid arthritis, asthma, allergic asthma, autoimmune hepatitis, primary sclerosing cholangitis, primary biliary cirrhosis, cirrhosis, neuromyelitis optica spectrum disorder (Devic's disease, opticospinal multiple sclerosis (OSMS)), pemphigus vulgaris, inflammatory bowel disease (IBD), systemic lupus erythematosus (SLE), celiac disease, psoriasis, autoimmune cardiomyopathy, idiopathic dilated cardiomyopathy (IDCM), myasthenia gravis, uveitis, ankylosing spondylitis, Immune Mediated Myopathies, anti-phospholipid syndrome (ANCA+), atherosclerosis, dermatomyositis, chronic obstructive pulmonary disease (COPD), emphysema, spinal cord injury, ANCA-associated vasculitis, idiopathic pulmonary fibrosis, pulmonary hypertension, non-alcoholic fatty liver disease (NAFLD), non-alcoholic steatohepatitis (NASH) or psoriasis. In certain embodiments, the autoimmune or inflammatory disorder comprises autoimmune hepatitis, primary sclerosing cholangitis, primary biliary cirrhosis, non-alcoholic fatty liver disease (NAFLD), non-alcoholic steatohepatitis (NASH) or cirrhosis. In certain embodiments, described herein, is a method of creating or expanding immunoregulatory invariant NKT cells comprising contacting the non-classical MHC-nanoparticles, described herein, or a composition comprising the non-classical MHC-nanoparticles to an invariant NKT cell. In certain embodiments, the contacting is in vitro. In certain embodiments, described herein, is a method of creating or expanding immunoregulatory invariant NKT cells in an individual comprising administering to the individual the non-classical MHC-nanoparticle or a composition comprising the non-classical MHC-nanoparticles. In certain embodiments, the immunoregulatory invariant NKT cells express MAF. In certain embodiments, the immunoregulatory invariant NKT cells express IL-10 and/or IL-21. In certain embodiments, the immunoregulatory invariant NKT cells express any one or more of LAG3, CTLA4, SLAMF6 and ITGA4. In certain embodiments, the immunoregulatory invariant NKT cells are CD4+ invariant NKT cells.

### BRIEF DESCRIPTION OF THE DRAWINGS

FIG. 1 illustrates a canonical non-classical MHC molecule, CD1d, comprising an NKT cell ligand coupled to a nanoparticle of the current disclosure. FIG. 1 is not to scale.

FIG. 2 illustrates the effect of treatment with αGalCer (KRN7000)/CD1d-coated NPs vs soluble KRN7000 or uncoated NPs on readouts of PBC in NOD.c3c4 mice. CBD, Common Bile Duct.

FIG. 3 illustrates the effect of treatment with αGalCer (KRN7000)/CD1d-coated NPs vs uncoated NPs on the CD4/CD8 phenotype of liver (top) and splenic (bottom) iNKT cells (stained with αGalCer (KRN7000)/CD1d tetramers).

FIG. 4 illustrates the effect of treatment with αGalCer (KRN7000)/CD1d-coated NPs vs uncoated NPs on the levels of PD1, CD49d, CD69 and LAG3 in liver iNKT cells (stained with αGalCer (KRN7000)/CD1d tetramers).

FIG. 5 illustrates normalized RNA expression counts (±SEM) for iNKT-relevant genes in the liver iNKT cells of NOD.c3c4 mice treated with αGalCer (KRN7000)/CD1d-NPs vs. control NOD.c3c4 mice.

FIG. 6A illustrates normalized RNA expression counts (±SEM) for genes described to be upregulated in iNKT1 cells, in the liver iNKT cells of NOD.c3c4 mice treated with αGalCer (KRN7000)/CD1d-NPs (right bar) vs. control NOD.c3c4 mice (left bar).

FIG. 6B illustrates normalized RNA expression counts (±SEM) for iNKT2-enriched genes in the liver iNKT cells of NOD.c3c4 mice treated with αGalCer (KRN7000)/CD1d-NPs (right bar) vs. control NOD.c3c4 mice (left bar).

FIG. 6C illustrates normalized RNA expression counts (±SEM) for iNKT10-enriched genes in the liver iNKT cells of NOD.c3c4 mice treated with αGalCer (KRN7000)/CD1d-NPs (right bar) vs. control NOD.c3c4 mice (left bar).

FIG. 6D illustrates normalized RNA expression counts (±SEM) for iNKT17-enriched genes in the liver iNKT cells of NOD.c3c4 mice treated with αGalCer (KRN7000)/CD1d-NPs (right bar) vs. control NOD.c3c4 mice (left bar).

FIG. 7 illustrates co-regulation of genes upregulated in the liver iNKT cells of mice treated with αGalCer (KRN7000)7CD1d-NPs vs. liver iNKT cells from control mice, by three transcription factors upregulated in response to treatment (MAF, GATA-3 and VDR). Shown is a Perfuse Force Directed cluster map.

FIG. 8 illustrates a comparison of the normalized RNA counts for 66 genes in liver iNKT cells induced by αGalCer/CD1d-NPs and splenic TR1-like CD4+ T-cells induced by pMHC class II-NPs. The 66 genes selected for comparison correspond to those that are differentially expressed with >4-fold difference in the liver iNKT cells of αGalCer/CD1d-NP-treated NOD.c3c4 mice vs. control NOD.c3c4 mice (from a total of 238 differentially expressed genes) and that are also differentially expressed in the TR1-like CD4+ T-cells induced by pMHC class II-coated NPs as compared to conventional CD4+ T-cells. The magnitude of expression of these genes is similar in iNKT vs TR1 cells induced by pMHC-NP treatment.

FIG. 9 illustrates that liver iNKT cells from αGalCer/CD1d-NP-treated mice could suppress the ability of CD11b+ APCs isolated from the liver-draining LNs and liver Kupffer cells from untreated mice, to present a model antigenic peptide (IGRP206-214) to cognate TCR-transgenic CD8+ T-cells.

FIG. 10 illustrates that liver iNKT cells from αGalCer/CD1d-NP-treated NOD.c3c4 mice could transfer disease protection to NOD.c3c4.scid mice reconstituted with splenocytes from diseased NOD.c3c4 donors, as compared to liver iNKT cells from control NOD.c3c4 donors.

FIG. 11 illustrates that liver and splenic iNKT cells from αGalCer/CD1d-NP-treated NOD.c3c4 mice, unlike those isolated from control mice, can trigger the conversion of conventional (IL-10/eGFP-negative) B-cells from NOD.I110eGFP donors (carrying eGFP in the I110 locus) into IL-10/eGFP-expressing CD1d-high/CD5+ B_{reg} cells within 7 days of transfer, even when the transferred B-cells were not pulsed with αGalCer, indicating that this process is driven by recognition of endogenous lipids presented by CD1d on B-cells.

FIG. 12 shows a non-limiting schematic of a polypeptide produced by a nucleic acid vector that can be used in making the non-classical MHC molecules of the description (SEQ ID NO: 6.

### DETAILED DESCRIPTION

Described herein is a non-classical MHC-nanoparticle comprising: (a) a sphingolipid or an analog thereof; (b) a non-classical MHC molecule; and (c) and a nanoparticle; wherein the sphingolipid or an analog thereof is associated with a binding groove of a non-classical MHC molecule, and wherein the non-classical MHC molecule is coupled to the nanoparticle. In certain embodiments, a plurality of the non-classical MHC-nanoparticles is included with a pharmaceutically acceptable carrier, diluent or excipient in a composition sufficiently pure for administration to an individual. In certain embodiments, the non-classical MHC-nanoparticle or the composition is for use in a method of creating, activating, expanding, or developing immunoregulatory invariant NKT cells. In certain embodiments, the immunoregulatory invariant NKT cells are CD4+ invariant NKT cells. In certain embodiments, the immunoregulatory invariant CD4+ NKT cells express MAF.

Described herein is a non-classical MHC-nanoparticle comprising: (a) a sphingolipid or an analog thereof; (b) a non-classical MHC molecule; and (c) and a nanoparticle; wherein the sphingolipid or an analog thereof is associated with a binding groove of a non-classical MHC molecule, and wherein the non-classical MHC molecule is coupled to the nanoparticle, wherein the non-classical MHC-NP does not comprise an immune activating co-stimulatory molecule or cytokine/cytokine receptor. In certain embodiments, a plurality of the non-classical MHC-nanoparticles is included with a pharmaceutically acceptable carrier, diluent or excipient in a composition sufficiently pure for administration to an individual. In certain embodiments, the non-classical MHC-nanoparticle or the composition is for use in a method of creating, activating, expanding, or developing immunoregulatory invariant NKT cells. In certain embodiments, the immunoregulatory invariant NKT cells are CD4+ invariant NKT cells. In certain embodiments, the immunoregulatory invariant CD4+ NKT cells express MAF.

Described herein is a non-classical MHC-nanoparticle comprising: (a) a sphingolipid or an analog thereof; (b) a non-classical MHC molecule; and (c) and a nanoparticle; wherein the sphingolipid or an analog thereof is associated with a binding groove of a non-classical MHC molecule, and wherein the non-classical MHC molecule is coupled to the nanoparticle, wherein the sphingolipid comprises an alpha-galactosylceramide. In certain embodiments, the alpha-galactosylceramide comprises KRN7000. In certain embodiments, a plurality of the non-classical MHC-nanoparticles is included with a pharmaceutically acceptable carrier, diluent or excipient in a composition sufficiently pure for administration to an individual. In certain embodiments, the non-classical MHC-nanoparticle or the composition is for use in a method of creating, activating, expanding, or developing immunoregulatory invariant NKT cells. In certain embodiments, the immunoregulatory invariant NKT cells are CD4+ invariant NKT cells. In certain embodiments, the immunoregulatory invariant CD4+ NKT cells express MAF.

Described herein is a non-classical MHC-nanoparticle comprising: (a) a sphingolipid or an analog thereof; (b) a non-classical MHC molecule; and (c) and a nanoparticle; wherein the sphingolipid or an analog thereof is associated with a binding groove of a non-classical MHC molecule, and wherein the non-classical MHC molecule is coupled to the nanoparticle, wherein the non-classical MHC-NP does not comprise an immune activating co-stimulatory molecule or cytokine/cytokine receptor, wherein the sphingolipid comprises an alpha-galactosylceramide. In certain embodiments, the alpha-galactosylceramide comprises KRN7000. In certain embodiments, a plurality of the non-classical MHC-nanoparticles is included with a pharmaceutically acceptable carrier, diluent or excipient in a composition sufficiently pure for administration to an individual. In certain embodiments, the non-classical MHC-nanoparticle or the composition is for use in a method of creating, activating, expanding, or developing immunoregulatory invariant NKT cells. In certain embodiments, the immunoregulatory invariant NKT cells are CD4+ invariant NKT cells. In certain embodiments, the immunoregulatory invariant CD4+ NKT cells express MAF.

Described herein is a non-classical MHC-nanoparticle comprising: (a) a sphingolipid or an analog thereof; (b) a non-classical MHC molecule; and (c) and a nanoparticle; wherein the sphingolipid or an analog thereof is associated with a binding groove of a non-classical MHC molecule, and wherein the non-classical MHC molecule is coupled to the nanoparticle. In certain embodiments, a plurality of the non-classical MHC-nanoparticles is included with a pharmaceutically acceptable carrier, diluent or excipient in a composition sufficiently pure for administration to an individual. In certain embodiments, the non-classical MHC-nanoparticle or the composition is for use in a method of treating an autoimmune or inflammatory condition in an individual. In certain embodiments, the autoimmune or inflammatory condition is an autoimmune or inflammatory condition that afflicts the liver.

Described herein is a non-classical MHC-nanoparticle comprising: (a) a sphingolipid or an analog thereof; (b) a non-classical MHC molecule; and (c) and a nanoparticle; wherein the sphingolipid or an analog thereof is associated with a binding groove of a non-classical MHC molecule, and wherein the non-classical MHC molecule is coupled to the nanoparticle, wherein the non-classical MHC-NP does not comprise an immune activating co-stimulatory molecule or cytokine/cytokine receptor. In certain embodiments, a plurality of the non-classical MHC-nanoparticles is included with a pharmaceutically acceptable carrier, diluent or excipient in a composition sufficiently pure for administration to an individual. In certain embodiments, the non-classical MHC-nanoparticle or the composition is for use in a method of treating an autoimmune or inflammatory condition in an individual. In certain embodiments, the autoimmune or inflammatory condition is an autoimmune or inflammatory condition that afflicts the liver.

Described herein is a non-classical MHC-nanoparticle comprising: (a) a sphingolipid or an analog thereof; (b) a non-classical MHC molecule; and (c) and a nanoparticle; wherein the sphingolipid or an analog thereof is associated with a binding groove of a non-classical MHC molecule, and wherein the non-classical MHC molecule is coupled to the nanoparticle, wherein the sphingolipid comprises an alpha-galactosylceramide. In certain embodiments, the alpha-galactosylceramide comprises KRN7000. In certain embodiments, a plurality of the non-classical MHC-nanoparticles is included with a pharmaceutically acceptable carrier, diluent or excipient in a composition sufficiently pure for administration to an individual. In certain embodiments, the non-classical MHC-nanoparticle or the composition is for use in a method of treating an autoimmune or inflammatory condition in an individual. In certain embodiments, the autoimmune or inflammatory condition is an autoimmune or inflammatory condition that afflicts the liver.

Described herein is a non-classical MHC-nanoparticle comprising: (a) a sphingolipid or an analog thereof; (b) a non-classical MHC molecule; and (c) and a nanoparticle; wherein the sphingolipid or an analog thereof is associated with a binding groove of a non-classical MHC molecule, and wherein the non-classical MHC molecule is coupled to the nanoparticle, wherein the non-classical MHC-NP does not comprise an immune activating co-stimulatory molecule or cytokine/cytokine receptor, wherein the sphingolipid comprises an alpha-galactosylceramide. In certain embodiments, the alpha-galactosylceramide comprises KRN7000. In certain embodiments, a plurality of the non-classical MHC-nanoparticles is included with a pharmaceutically acceptable carrier, diluent or excipient in a composition sufficiently pure for administration to an individual. In certain embodiments, the non-classical MHC-nanoparticle or the composition is for use in a method of treating an autoimmune or inflammatory condition in an individual. In certain embodiments, the autoimmune or inflammatory condition is an autoimmune or inflammatory condition that afflicts the liver.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

“Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. Compositions for treating or preventing a given disease can consist essentially of the recited active ingredient, exclude additional active ingredients, but include other non-active components such as excipients, carriers, or diluents. “Consisting of” shall mean excluding more than trace elements of other ingredients and substantial method steps. Embodiments defined by each of these transition terms are within the scope of this disclosure.

As used herein the term “about” refers to an amount that is near the stated amount by 10%.

As used herein the terms “individual,” “patient,” or “subject” are used interchangeably and refer to individuals diagnosed with, suspected of being afflicted with, or at-risk of developing at least one disease for which the described compositions and method are useful for treating. In certain embodiments, the individual is a mammal. In certain embodiments, the mammal is a mouse, rat, rabbit, dog, cat, horse, cow, sheep, pig, goat, llama, alpaca, or yak. In certain embodiments, the individual is a human.

As used herein the term “treat” or “treating” refers to interventions to a physiological or disease state of an individual designed or intended to ameliorate at least one sign or symptom associated with said physiological or disease state. The skilled artisan will recognize that given a heterogeneous population of individuals afflicted with a disease, not all individuals will respond equally, or at all, to a given treatment. With respect to treatments that effect the liver treatments can reduce or stabilize the levels of one or more liver enzymes selected from alanine transaminase (ALT), aspartate transaminase (AST), alkaline phosphatase (ALP), and gamma-glutamyl transpeptidase (GGT) as determined by standard lab tests. Liver treatments may also reduce or stabilize the amount of fibrosis of a liver, the amount of lipid stored in the liver, the size and/or weight of the liver as determined by MRI or biopsy. Treatments that affect the liver can reduce the amount of inflammatory immune cell infiltrate, reduce the amount of inflammatory cytokines (e.g., IFNgamma) or chemokines, increase the amount of suppressive immune regulatory cells such as suppressive invariant NKT cells, or increase the amount of suppressive cytokines such as IL-10.

“Particle,” or “nanoparticle,” as used herein is meant to describe small discrete particles that are administrable to a subject. In certain embodiments, the particles are substantially spherical in shape. The term “substantially spherical,” as used herein, means that the shape of the particles does not deviate from a sphere by more than about 10%. Various known antigen or polypeptide complexes of the disclosure may be applied to the particles. The particles or nanoparticles, described herein, may comprise a core fabricated from a first material, and optionally a layer fabricated from a second material. These layers may possess properties that enhance the overall stability, solubility, or bioavailability of the particle.

The terms “polypeptide” and “protein” are used interchangeably to refer to a polymer of amino acid residues, and are not limited to a minimum length. Polypeptides, including the provided non-classical MHC and non-classical MHC polypeptide chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The polypeptides, described herein, can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

The nucleic acids described herein can be stored or transported in a “master” cell bank. A master cell bank comprises a nucleic acid encoding any of the non-classical MHC molecules described herein contained within a suitable cell-line, and a cryo-preservative. In certain embodiments, the cell line is a bacterial cell line (e.g., E. coli). In certain embodiments, the cell line is a mammalian cell line (e.g., Chinese Hamster Ovary (CHO) cells, or Human Embryonic Kidney (HEK) cells). Suitable cryoprotectants comprise, for example, glycerol (about 10% to about 20%) or DMSO (about 10% to about 20%). The master cell bank can be stored in a suitable container able to withstand freezing to about −80° C. or about 196° C.

### Non-classical MHC Molecules

As used herein “non-classical MHC molecule” or “non-classical MHC” refers to MHC molecules other than classical MHC class I or MHC class II molecules. In certain embodiments, the non-classical MHC molecule comprises CD1d. In certain embodiments, the non-classical MHC molecule comprises human CD1d.

Described herein are nanoparticles coupled to non-classical MHC molecules. In certain embodiments, the non-classical MHC molecule coupled to the nanoparticle comprises a CD1d polypeptide associated with a binding cleft of the CD1d polypeptide. In certain embodiments, the non-classical MHC molecule coupled to the nanoparticle consists essentially of a CD1d polypeptide. In certain embodiments, the non-classical MHC molecule coupled to the nanoparticle consists of a CD1d polypeptide. CD1d proteins are largely invariant compared to classical MHC molecules, which are highly allelic. The CD1d protein is encoded by the human CD1D gene. In certain embodiments, the CD1d polypeptides, described herein, are human CD1d polypeptides at least about 90%, 95%, 97%, 98%, 99%, or 100% identical to a human CD1d polypeptide (SEQ ID NO: 1). In certain embodiments, the CD1d polypeptide comprises a polypeptide at least about 50, 75, 100, 150, 200, 250, 300, or more amino acids in length. In certain embodiments, the pathogen-associated antigen comprises a polypeptide less than about 100, 150, 200, 250, 300, 350 amino acids in length. When produced in an appropriate cellular system the CD1d will comprises a suitable signal sequence such as the endogenous signal sequence or a heterologous signal sequence. Such signal sequence will be cleaved and removed in the endoplasmic reticulum. In certain embodiments, the CD1d molecule comprises a signal sequence cleaved CD1d molecule at least about 90%, 95%, 97%, 98%, 99%, or 100% identical to a human CD1d polypeptide (SEQ ID NO: 2). Different production systems may result in further N-terminal trimming resulting in removal of 1, 2, 3, 4, 5, 6, 7, 8, 9, 10 amino acids or more from the N-terminus of SEQ ID NO: 2. The CD1d molecule utilized herein may also comprise deletions of a transmembrane domain, an intracellular domain, or both. In certain embodiments, the CD1d molecule comprises a signal sequence cleaved, transmembrane/intracellular domain deleted CD1d molecule at least about 90%, 95%, 97%, 98%, 99%, or 100% identical to a human CD1d polypeptide (SEQ ID NO: 3). The sequence of CD1d can further comprise a deletion of 1, 2, 3, 4, 5, 6, 7, 8, 9, 10, 11, 12, 13, 14, 15, 16, 17, 18, 19, 20 or more amino acids from the N- or C-terminus of SEQ ID NO: 3. In certain embodiments, the CD1d molecule comprises a signal sequence cleaved, transmembrane/intracellular domain deleted CD1d molecule at least about 90%, 95%, 97%, 98%, 99%, or 100% identical to a human CD1d polypeptide (SEQ ID NO: 4). The sequence of CD1d can further comprise a deletion of 1, 2, 3, 4, 5, 6, 7, 8, 9, 10, 11, 12, 13, 14, 15, 16, 17, 18, 19, 20 or more amino acids from the N- or C-terminus of SEQ ID NO: 4. In certain embodiments, the CD1d polypeptide is about 90% to 100% identical, or is 90% to 100% identical, to SEQ ID NO: 1, 2, 3, or 4. In certain embodiments, the CD1d polypeptide is about 90% to about 91%, about 90% to about 92%, about 90% to about 93%, about 90% to about 94%, about 90% to about 95%, about 90% to about 96%, about 90% to about 97%, about 90% to about 98%, about 90% to about 99%, about 90% to 100%, about 91% to about 92%, about 91% to about 93%, about 91% to about 94%, about 91% to about 95%, about 91% to about 96%, about 91% to about 97%, about 91% to about 98%, about 91% to about 99%, about 91% to 100%, about 92% to about 93%, about 92% to about 94%, about 92% to about 95%, about 92% to about 96%, about 92% to about 97%, about 92% to about 98%, about 92% to about 99%, about 92% to 100%, about 93% to about 94%, about 93% to about 95%, about 93% to about 96%, about 93% to about 97%, about 93% to about 98%, about 93% to about 99%, about 93% to 100%, about 94% to about 95%, about 94% to about 96%, about 94% to about 97%, about 94% to about 98%, about 94% to about 99%, about 94% to 100%, about 95% to about 96%, about 95% to about 97%. about 95% to about 98%, about 95% to about 99%, about 95% to 100%, about 96% to about 97%, about 96% to about 98%, about 96% to about 99%, about 96% to 100%, about 97% to about 98%, about 97% to about 99%, about 97% to 100%, about 98% to about 99%, about 98% to 100%, or about 99% to 100% identical to SEQ ID NO: 1, 2, 3, or 4. In certain embodiments, the CD1d polypeptide is about 90%, about 91%, about 92%, about 93%, about 94%, about 95%, about 96%, about 97%, about 98%, about 99%, or 100% identical to SEQ ID NO: 1, 2, 3, or 4. In certain embodiments, the CD1d polypeptide is at least about 90%, about 91%, about 92%, about 93%, about 94%, about 95%, about 96%, about 97%, about 98%, or about 99% identical to SEQ ID NO: 1, 2, 3, or 4. In certain embodiments, the CD1d polypeptide is at most about 91%, about 92%, about 93%, about 94%, about 95%, about 96%, about 97%, about 98%, about 99%, or 100% identical to SEQ ID NO: 1, 2, 3, or 4. In certain embodiments, the CD1d polypeptide is identical to any one of SEQ ID NO: 1, 2, 3, or 4.

In vivo Cd1d associates with β₂m to facilitate NKT cell engagement. Thus, the ncMHC-NP may also comprise a β₂m polypeptide. In certain embodiments, the CD1d polypeptides, described herein, are β₂m polypeptides at least about 90%, 95%, 97%, 98%, 99%, or 100% identical to a human β₂m polypeptide (SEQ ID NO: 5). In certain embodiments, the β₂m polypeptide comprises a polypeptide at least about 10, 20, 30, 40, 50, 75, 100, or more amino acids in length. In certain embodiments, the pathogen-associated antigen comprises a polypeptide less than about 100, 75, 50, 40, 30, 20, or 10 amino acids in length. The sequence of β₂m can further comprise a deletion of 1, 2, 3, 4, 5, 6, 7, 8, 9, 10, 11, 12, 13, 14, 15, 16, 17, 18, 19, 20 or more amino acids from the N- or C-terminus of SEQ ID NO: 5. The β₂m polypeptide can further be coupled to the Cd1d polypeptide by linker. In certain embodiments, the β₂m polypeptide can further be coupled to the N-terminus of the Cd1d polypeptide by linker. In certain embodiments, the β₂m polypeptide can further be coupled to the C-terminus of the Cd1d polypeptide by linker. In certain embodiments, the linker is a flexible polypeptide linker. Such linkers include a Gly-Ser linker (G₃S₁)_{X} or (G4S1)_{X}. In certain embodiments, X is any whole number integer such as 1, 2, 3, 4, or 5.

The CD1d-β₂m polypeptides can be expressed and purified as a single polypeptide. In certain embodiments, the CD1d-β₂m polypeptides, described herein, are human CD1d-β₂m polypeptides at least about 90%, 95%, 97%, 98%, 99%, or 100% identical to a human CD1d-β₂m polypeptide (SEQ ID NO: 6). In certain embodiments, the CD1d-β₂m polypeptides, described herein, comprise CD1d-β₂m polypeptides at least about 90%, 95%, 97%, 98%, 99%, or 100% identical to a CD1d-β₂m polypeptide (SEQ ID NO: 6 amino acids 1 to 427). In certain embodiments, described herein is a nucleic acid comprising a nucleic acid sequence encoding a polypeptide at least about 90%, 95%, 97%, 98%, 99%, or 100% identical to a CD1d-β₂m polypeptide (SEQ ID NO: 6 amino acids 1 to 427).

In certain embodiments, the CD1d polypeptide comprises a C-terminal cysteine or lysine to allow coupling to a functionalized linker or layer. In certain embodiments, the linker or layer is a functionalized PEG or dextran molecule.

As described herein, and shown by the applicant, NKT cells exist as many different subsets, some of which are inflammatory and some of which are regulatory or anti-inflammatory. Applicant has discovered that CD1d glycolipid antigens presented to NKT cells in the context of nanoparticles reprogram iNKT cells into a novel regulatory subset of iNKT cells that are useful for the treatment of autoimmune and inflammatory diseases.

CD1d presents glycolipid antigens to iNKT cells. The ncMHC-NP, described herein, comprise a glycolipid ligand bound to the binding cleft of the non-classical MHC molecule. In certain embodiments, the non-classical MHC of the ncMHC-NP comprises or consists of CD1d. In certain embodiments, the non-classical MHC-nanoparticle comprises a sphingolipid or an analog thereof. In certain embodiments, the sphingolipid or analog thereof comprises a ceramide or analog thereof. In certain embodiments, the ceramide or an analog thereof comprises alpha-galactosylceramide (KRN7000), alpha-C-galactosylceramide, alpha-glucuronosyl ceramide, beta-galactosylceramide, PBS-20, PBS-25, sulfatide, isoglobotriosylceramide (iGb3), GSL-1, α-glucuronosylceramide, phosphatidylinositol mannoside (PIM), PIM₄, phenyl pentamethyldihydrobenzofuransulfonate (PPBF), phosphatidylethanolamine (PE), phosphatidylcholine (PC) or combinations thereof. In certain embodiments, the ceramide or an analog thereof comprises alpha-galactosylceramide (KRN7000), alpha-C-galactosylceramide, alpha-glucuronosyl ceramide, beta-galactosylceramide, PBS-20, PBS-25, sulfatide, isoglobotriosylceramide (iGb3), or combinations thereof. In certain embodiments, in the ceramide or an analog thereof comprises alpha-galactosylceramide (αGalCer or KRN7000). In certain embodiments, the CD1d comprises one, two, three, four, five, or six different ceramide ligands. In certain embodiments, the CD1d comprises a single type of ceramide ligand. In certain embodiments, the ceramide ligand comprises αGalCer or KRN7000. In certain embodiments, the ceramide is derived from a bacterial ceramide of Sphingomonas (S. capsulata, S. paucimobilis, and S. wittichii) or Ehrlichia muris.

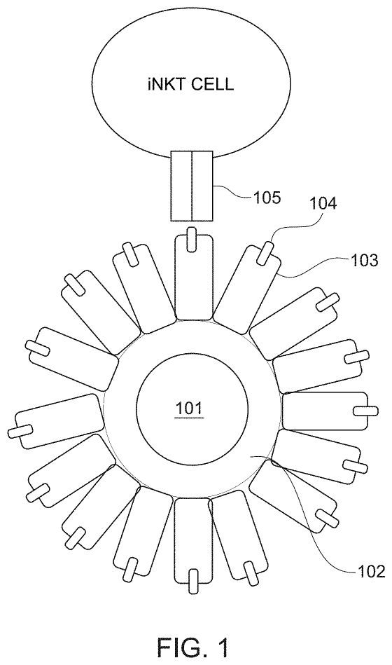

Referring to FIG. 1, a specific non-limiting embodiment of the ncMHC-NP described herein, comprises a core 101, optionally surrounded by a hydrophilic layer 102. The ncMHC further comprises a plurality of CD1d molecules 103 comprising a sphingolipid ligand 104. This ligand is associated to the binding cleft of the CD1d molecule such that an invariant alpha-beta T cell receptor 105 of an iNKT cell can interact with the ligand. The core 101 can suitably comprise a dense material, and in certain embodiments the dense material comprises a metal or metal oxide. The optional layer 102 can be a hydrophilic biocompatible material such as polyethylene glycol (PEG), dextran, or mannitol. The layer can further be functionalized such that a CD1d molecule 103 can be coupled to the layer. Skilled artisans will recognize that due to steric hindrance not every molecule of the layer will be or can be coupled to a CD1d molecule. Additionally, the layer, while substantially homogeneously coating the particle can increase the total diameter of a complex by at least about 5 nm, 10 nm, 15 nm, 20 nm, 25 nm, 30 nm, 40 nm, or 50 nm. Additionally, the layer can be a single type of hydrophilic molecule or mixture of different types with a first type conjugated to CD1d molecules, and a second type providing other properties that are desirable for stability, solubility, bioavailability, and/or bioabsorption. In certain embodiments, the ncMHC-NP does not comprise an immune activating co-stimulatory molecule or cytokine/cytokine receptor.

### Nanoparticles

The nanoparticles of the ncMHC-NP are suitably made of a dense non-liposomal material. The nanoparticle core of the ncMHC-NP comprises, or consists essentially of, or yet further consists of a core, for example a solid core, a metal core, a dendrimer core, a polymeric micelle nanoparticle core, a nanorod, a fullerene, a nanoshell, a coreshell, a protein-based nanostructure or a lipid-based nanostructure. In some aspects, the nanoparticle core is bioabsorbable and/or biodegradable. In some aspects, the nanoparticle core is a dendrimer nanoparticle core comprising, or alternatively consisting essentially thereof, or yet further consisting of a highly branched macromolecule having a tree-like structure growing from a core. In further aspects, the dendrimer nanoparticle core may comprise, or alternatively consist essentially thereof, or yet further consist of a poly(amidoamine)-based dendrimer or a poly-L-lysine-based dendrimer. In certain aspects, the nanoparticle core is a polymeric micelle core comprising, or alternatively consisting essentially thereof, or yet further consisting of an amphiphilic block co-polymer assembled into a nano-scaled core-shell structure. In further aspects, the polymeric micelle core comprises, or alternatively consists essentially thereof, or yet further consists of a polymeric micelle produced using polyethylene glycol-diastearoylphosphatidylethanolamine block copolymer. In a further aspect, the nanoparticle core comprises, or alternatively consists essentially of, or yet further consists of a metal. In another aspect, the nanoparticle core is not a liposome. Additional examples of core materials include but are not limited to, standard and specialty glasses, silica, polystyrene, polyester, polycarbonate, acrylic polymers, polyacrylamide, polyacrylonitrile, polyamide, fluoropolymers, silicone, celluloses, silicon, metals (e.g., iron, gold, silver), minerals (e.g., ruby), nanoparticles (e.g., gold nanoparticles, colloidal particles, metal oxides, metal sulfides, metal selenides, and magnetic materials such as iron oxide), and composites thereof. In some embodiments, an iron oxide nanoparticle core comprises or consists of iron (II, III) oxide. The core could be of homogeneous composition, or a composite of two or more classes of material depending on the properties desired. In certain aspects, metal nanoparticles will be used. These metal particles or nanoparticles can be formed from Au, Pt, Pd, Cu, Ag, Co, Fe, Ni, Mn, Sm, Nd, Pr, Gd, Ti, Zr, Si, and In, precursors, their binary alloys, their ternary alloys and their intermetallic compounds. See U.S. Pat. No. 6,712,997, which is incorporated herein by reference in its entirety. In certain embodiments, the compositions of the core and layers (described below) may vary provided that the nanoparticles are biocompatible and bioabsorbable. The core could be of homogeneous composition, or a composite of two or more classes of material depending on the properties desired. In certain aspects, metal nanospheres will be used. These metal nanoparticles can be formed from Fe, Ca, Ga and the like. In certain embodiments, the nanoparticle comprises, or alternatively consists essentially of, or yet further consists of a core comprising metal or metal oxide such as gold, iron, or iron oxide.

The size of the nanoparticle core can range from about 1 nm to about 1 μm. In certain embodiments, the nanoparticle core is less than about 1 μm in diameter. In other embodiments, the nanoparticle core is less than about 500 nm, less than about 400 nm, less than about 300 nm, less than about 200 nm, less than about 100 nm, less than about 90 nm, less than about 80 nm, less than about 75 nm, less than about 70 nm, less than about 60 nm, less than about 50 nm, less than about 40 nm, or less than about 30 nm in diameter. In other embodiments, the nanoparticle core is greater than about 5 nm, greater than about 6 nm, greater than about 7 nm, greater than about 7 nm, greater than about 8 nm, greater than about 9 nm, greater than about 10 nm, greater than about 12 nm, greater than about 15 nm, greater than about 16 nm, greater than about 17 nm greater than about 18 nm, greater than about 19 nm, greater than about 20 nm, or greater than about 21 nm in diameter. In certain embodiments, the nanoparticle core is from about 1 nm to about 10 nm, 15 nm, 20 nm, 25 nm, 30 nm, 40 nm, 50 nm, 75 nm, or 100 nm in diameter. In certain embodiments, the nanoparticle core is from about 5 nm to about 10 nm, 15 nm, 20 nm, 25 nm, 30 nm, 40 nm, 50 nm, 75 nm, or 100 nm in diameter. In certain embodiments, the nanoparticle core is from about 6 nm to about 10 nm, 15 nm, 20 nm, 25 nm, 30 nm, 40 nm, 50 nm, 75 nm, or 100 nm in diameter. In certain embodiments, the nanoparticle core is from about 7 nm to about 10 nm, 15 nm, 20 nm, 25 nm, 30 nm, 40 nm, 50 nm, 75 nm, or 100 nm in diameter. In certain embodiments, the nanoparticle core is from about 8 nm to about 15 nm, 20 nm, 25 nm, 30 nm, 40 nm, 50 nm, 75 nm, or 100 nm in diameter. In certain embodiments, the nanoparticle core is from about 9 nm to about 15 nm, 20 nm, 25 nm, 30 nm, 40 nm, 50 nm, 75 nm, or 100 nm in diameter. In certain embodiments, the nanoparticle core is from about 10 nm to about 15 nm, 20 nm, 25 nm, 30 nm, 40 nm, 50 nm, 75 nm, or 100 nm in diameter. In certain embodiments, the nanoparticle core is from about 11 nm to about 15 nm, 20 nm, 25 nm, 30 nm, 40 nm, 50 nm, 75 nm, or 100 nm in diameter. In certain embodiments, the nanoparticle core is from about 12 nm to about 20 nm, 25 nm, 30 nm, 40 nm, 50 nm, 75 nm, or 100 nm in diameter. In certain embodiments, the nanoparticle core is from about 13 nm to about 20 nm, 25 nm, 30 nm, 40 nm, 50 nm, 75 nm, or 100 nm in diameter. In certain embodiments, the nanoparticle core is from about 14 nm to about 20 nm, 25 nm, 30 nm, 40 nm, 50 nm, 75 nm, or 100 nm in diameter. In certain embodiments, the nanoparticle core is from about 15 nm to about 20 nm, 25 nm, 30 nm, 40 nm, 50 nm, 75 nm, or 100 nm in diameter. In certain embodiments, the nanoparticle core is from about 16 nm to about 20 nm, 25 nm, 30 nm, 40 nm, 50 nm, 75 nm, or 100 nm in diameter. In certain embodiments, the nanoparticle core is from about 17 nm to about 25 nm, 30 nm, 40 nm, 50 nm, 75 nm, or 100 nm in diameter. In certain embodiments, the nanoparticle core is from about 18 nm to about 25 nm, 30 nm, 40 nm, 50 nm, 75 nm, or 100 nm in diameter. In certain embodiments, the nanoparticle core is from about 19 nm to about 25 nm, 30 nm, 40 nm, 50 nm, 75 nm, or 100 nm in diameter. In certain embodiments, the nanoparticle core is from about 20 nm to about 25 nm, 30 nm, 40 nm, 50 nm, 75 nm, or 100 nm in diameter. In certain embodiments, the nanoparticle core is from about 21 nm to about 25 nm, 30 nm, 40 nm, 50 nm, 75 nm, or 100 nm in diameter.

In specific embodiments, the nanoparticle core has a diameter of from about 1 nm to about 100 nm; from about 1 nm to about 75 nm; from about 1 nm to about 50 nm; from about 1 nm to about 25 nm; from about 1 nm to about 25 nm; from about 5 nm to about 100 nm; from about 5 nm to about 50 nm; from about 5 nm to about 40 nm; from about 5 nm to about 30 nm; from about 5 nm to about 25 nm, or from about 5 nm to about 20 nm. In some embodiments, the nanoparticles core has a diameter of from about 10 nm to about 60 nm, from about 10 nm to about 50 nm, from about 10 nm to about 40 nm, from about 10 nm to about 30 nm, from about 10 nm to about 25nm, from about 10 nm to about 20 nm. In some embodiments, the nanoparticles core has a diameter of from about 15 nm to about 50 nm, from about 15 nm to about 40 nm, from about 15 nm to about 35 nm, from about 15 nm to about 30 nm, or from about 15 nm to about 25 nm.

The nanoparticles typically consist of a substantially spherical core and optionally one or more layers or coatings. The core may vary in size and composition as described herein. In addition to the core, the particle may have one or more layers to provide functionalities appropriate for the applications of interest. Layers may impart chemical or biological functionalities, referred to herein as chemically active or biologically active layers. These layers typically are applied on the outer surface of the particle and can impart functionalities to the ncMHC-NPs. The layer or layers may typically range in thickness from about 1 to 100 nm. The thickness of a layer may vary from about 1 nm to 5 nm, from about 1 nm to about 10 nm, from about 1 nm to about 40 nm, or from about 1 nm to about 50 nm. In certain embodiments, the layer may be adsorbed to the nanoparticle core. In certain embodiments, the layer may be covalently coupled to the nanoparticle core using any appropriate chemistry based upon the core composition. For example, if the nanoparticle comprises a metal or metal oxide the layer may be coupled to the nanoparticle core via a coordinate covalent bond.

The layer or coating may comprise, or alternatively consist essentially of, or yet further consist of a biodegradable sugar or other polymer. Examples of biodegradable layers include but are not limited to dextran; poly(ethylene glycol) (PEG); poly(ethylene oxide); mannitol; poly(esters) based on polylactide (PLA), polyglycolide (PGA), polycaprolactone (PCL); poly(hydroxalkanoate) of the PHB-PHV class; and other modified poly(saccharides) such as starch, cellulose and chitosan. Additionally, the nanoparticle may include a layer with suitable surfaces for attaching chemical functionalities for chemical binding or coupling sites.

Layers can be produced on the nanoparticles in a variety of ways known to those skilled in the art. Examples include sol-gel chemistry techniques such as described in Iler, Chemistry of Silica, John Wiley & Sons, 1979; Brinker and Scherer, Sol-gel Science, Academic Press, (1990). Additional approaches to producing layers on nanoparticles include surface chemistry and encapsulation techniques such as described in Partch and Brown, J. Adhesion, 67:259-276, 1998; Pekarek et al., Nature, 367:258, (1994); Hanprasopwattana, Langmuir, 12:3173-3179, (1996); Davies, Advanced Materials, 10:1264-1270, (1998); and references therein. Vapor deposition techniques may also be used; see, for example, Golman and Shinohara, Trends Chem. Engin., 6:1-6, (2000); and U.S. Pat. No. 6,387,498. Still other approaches include layer-by-layer self-assembly techniques such as described in Sukhorukov et al., Polymers Adv. Tech., 9 (10-11):759-767, (1998); Caruso et al., Macromolecules, 32 (7):2317-2328, (1998); Caruso et al., J. Amer. Chem. Soc., 121 (25):6039-6046, (1999); U.S. Pat. No. 6,103,379 and references cited therein.

A plurality of non-classical MHC molecules can be coupled to a nanoparticle at a particular valency. Valency is the number of ncMHC complexes per nanoparticle core. In certain embodiments, the valency of the nanoparticle may range between about 1 ncMHC complex to 1 per nanoparticle core (1:1) to about 6000 ncMHC complexes per 1 nanoparticle core (6000:1). In some embodiments, the valency is from about 1:1 to about 6000:1, from about 1:1 to about 5500:1, from about 1:1 to about 5000:1, from about 1:1 to about 4000:1, from about 1:1 to about 3500:1, from about 1:1 to about 3000:1, from about 1:1 to about 2500:1, from about 1:1 to about 2000:1, from about 1:1 to about 1500:1, from about 1:1 to 1000:1, from about 1:1 to about 500:1, from about 1:1 to about 400:1, from about 1:1 to about 300:1, from about 1:1 to about 200:1, from about 1:1 to about 100:1, or from about 1:1 to about 50:1. In some embodiments, the valency is from about 2:1 to about 6000:1, from about 2:1 to about 5500:1, from about 2:1 to about 5000:1, from about 2:1 to about 4000:1, from about 2:1 to about 3500:1, from about 2:1 to about 3000:1, from about 2:1 to about 2500:1, from about 2:1 to about 2000:1, from about 2:1 to about 1500:1, from about 2:1 to 1000:1, from about 2:1 to about 500:1, from about 2:1 to about 400:1, from about 2:1 to about 300:1, from about 2:1 to about 200:1, from about 2:1 to about 100:1, or from about 2:1 to about 50:1. In some embodiments, the valency is from about 5:1 to about 6000:1, from about 5:1 to about 5500:1, from about 5:1 to about 5000:1, from about 5:1 to about 4000:1, from about 5:1 to about 3500:1, from about 5:1 to about 3000:1, from about 5:1 to about 2500:1, from about 5:1 to about 2000:1, from about 5:1 to about 1500:1, from about 5:1 to 1000:1, from about 5:1 to about 500:1, from about 5:1 to about 400:1, from about 5:1 to about 300:1, from about 5:1 to about 200:1, from about 5:1 to about 100:1, or from about 5:1 to about 50:1. In some embodiments, the valency is from about 8:1 to about 6000:1, from about 8:1 to about 5500:1, from about 8:1 to about 5000:1, from about 8:1 to about 4000:1, from about 8:1 to about 3500:1, from about 8:1 to about 3000:1, from about 8:1 to about 2500:1, from about 8:1 to about 2000:1, from about 8:1 to about 1500:1, from about 8:1 to 1000:1, from about 8:1 to about 500:1, from about 8:1 to about 400:1, from about 8:1 to about 300:1, from about 8:1 to about 200:1, from about 8:1 to about 100:1, or from about 8:1 to about 50:1. In some aspects, the valency is from about 10:1 to about 6000:1, from about 20:1 to about 5500:1, from about 10:1 to about 5000:1, from about 10:1 to about 4000:1, from about 10:1 to about 3500:1, from about 10:1 to about 3000:1, from about 10:1 to about 2500:1, from about 10:1 to about 2000:1, from about 10:1 to about 1500:1, from about 10:1 to 1000:1, from about 10:1 to about 500:1, from about 10:1 to about 400:1, from about 10:1 to about 300:1, from about 10:1 to about 200:1, from about 10:1 to about 100:1, or from about 10:1 to about 50:1. In some embodiments, the valency is from about 15:1 to about 6000:1, from about 15:1 to about 5500:1, from about 15:1 to about 5000:1, from about 15:1 to about 4000:1, from about 15:1 to about 3500:1, from about 15:1 to about 3000:1, from about 15:1 to about 2500:1, from about 15:1 to about 2000:1, from about 15:1 to about 1500:1, from about 15:1 to 1000:1, from about 15:1 to about 500:1, from about 15:1 to about 400:1, from about 15:1 to about 300:1, from about 15:1 to about 200:1, from about 15:1 to about 100:1, or from about 15:1 to about 50:1. In some embodiments, the valency is from about 20:1 to about 6000:1, from about 20:1 to about 5500:1, from about 20:1 to about 5000:1, from about 20:1 to about 4000:1, from about 20:1 to about 3500:1, from about 20:1 to about 3000:1, from about 20:1 to about 2500:1, from about 20:1 to about 2000:1, from about 20:1 to about 1500:1, from about 20:1 to 1000:1, from about 20:1 to about 500:1, from about 20:1 to about 400:1, from about 20:1 to about 300:1, from about 20:1 to about 200:1, from about 20:1 to about 100:1, or from about 20:1 to about 50:1.

### Methods of Making Non-classical MHC Molecules

Nanoparticles may be formed by contacting an aqueous phase containing the ncMHC complex, and a polymer and a nonaqueous phase followed by evaporation of the nonaqueous phase to cause the coalescence of particles from the aqueous phase as taught in U.S. Pat. Nos. 4,589,330 or 4,818,542. Certain polymers for such preparations are natural or synthetic copolymers or polymers which include gelatin agar, starch, arabinogalactan, albumin, collagen, polyglycolic acid, polylactic acid, glycolide-L(-) lactide poly(episilon-caprolactone, poly(epsilon-caprolactone-CO-lactic acid), poly(epsilon-caprolactone-CO-glycolic acid), poly(β-hydroxy butyric acid), poly(ethylene oxide), polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) and poly(methyl methacrylate). Particularly, certain polymers are polyesters, such as polyglycolic acid, polylactic acid, glycolide-L(-) lactide poly(episilon-caprolactone), poly(epsilon-caprolactone-CO-lactic acid), and poly(epsilon-caprolactone-CO-glycolic acid). Solvents useful for dissolving the polymer include: water, hexafluoroisopropanol, methylenechloride, tetrahydrofuran, hexane, benzene, or hexafluoroacetone sesquihydrate.

The ncMHC described herein can be coupled to a nanoparticle via the layers previously mentioned or if no layer is present a linker molecule. In certain embodiments, such linker molecules comprise, consist essentially of, or consist of polyethylene glycol (PEG), dextran, or mannitol. In certain embodiments, such linker molecules comprise, consist essentially of, or consist of polyethylene glycol (PEG). In certain embodiments, such linker molecules comprise, consist essentially of, or consist of dextran. Such layers and linkers can be functionalized or derivatized with a group that is able to form a covalent bond with the ncMHC. The reaction can be any suitable reaction, including but not limited to, an amine-to-amine, sulfhydryl-to-sulfhydryl, amine-to-sulfhydryl, carboxyl-to-amine, or sulfhydryl-to-carboxyl. In certain embodiments, the ncMHC is coupled to the linker or layer by the reaction of an NHS ester and a primary amine on the ncMHC (e.g., a lysine residue). In certain embodiments, the ncMHC is coupled to the linker or layer by the reaction of an imidoester and a primary amine on the ncMHC (e.g., a lysine residue). In certain embodiments, the ncMHC is coupled to the linker or layer by the reaction of an amide group and a sulfhydryl group (e.g., cysteine residue) on the ncMHC. In certain embodiments, the ncMHC is coupled to the linker or layer by the reaction of a maleimide group and a sulfhydryl group (e.g., cysteine residue) on the ncMHC. In certain embodiments, the ncMHC is coupled to the linker or layer by the reaction of a maleimide group and a primary amine group on ncMHC. In certain embodiments, the ncMHC is coupled to the linker or layer by the reaction of a haloacetyl group and a sulfhydryl group on ncMHC. In certain embodiments, the ncMHC is coupled to the linker or layer by the reaction of a haloacetyl group and a primary amine group on the ncMHC. In certain embodiments, the ncMHC is coupled to the linker or layer by the reaction of a pyridyldithiol and a sulfhydryl group on ncMHC. In certain embodiments, the ncMHC is coupled to the linker or layer by the reaction of a pyridyldithiol and a primary amine group on the ncMHC. In certain embodiments, the ncMHC is coupled to the linker or layer by the reaction of a carbodiimide and a primary amine group on ncMHC. In certain embodiments, the ncMHC is coupled to the linker or layer by a heterobifunctional linker. In certain embodiments, the ncMHC is coupled to the linker or layer by the reaction of a maleimide/hydrazide and a sulfhydryl group on the ncMHC. In certain embodiments, the ncMHC is coupled to the linker or layer by the reaction of a pyridyldithiol/hydrazide and a sulfhydryl group on the ncMHC. In certain embodiments, the crosslinker is a photoreactive crosslinker.

Gold nanoparticles (GNPs) are synthesized using chemical reduction of gold chloride with sodium citrate as described (Perrault, S. D. et al. (2009) Nano Lett 9:1909-1915). Briefly, 2 mL of 1% of HAuCl4 (Sigma Aldrich) is added to 100 mL H2O under vigorous stirring and the solution is heated in an oil bath. Six (for 14 nm GNPs) or two mL (for 40 nm GNPs) of 1% Na Citrate is added to the boiling HAuCl4 solution, which is stirred for an additional 10 min and then is cooled down to room temperature. GNPs are stabilized by the addition of 1 μMol of thiol-PEG linkers (Nanocs, Mass.) functionalized with —COOH or —NH2 groups as acceptors of MHC. Pegylated GNPs are washed with water to remove free thiol-PEG, concentrated and stored in water for further analysis. NP density is determined via spectrophotometry and calculated according to Beer's law.

The iron oxide NPs (SFP IONPs) can also be produced by thermal decomposition of an iron salt (such as iron acetate) in organic solvents in the presence of surfactants, then rendered solvent in aqueous buffers by pegylation (Xie, J. et al. (2007) Adv Mater 19:3163; Xie, J. et al. (2006) Pure Appl. Chem. 78:1003-1014; Xu, C. et al. (2007) Polymer International 56:821-826). Briefly, 2 mMol Fe(acac)₃ (Sigma Aldrich, Oakville, ON) are dissolved in a mixture of 10 mL benzyl ether and oleylamine and heated to 100° C. for 1 hour followed by 300° C. for 2 hours with reflux under the protection of a nitrogen blanket. Synthesized NPs are precipitated by addition of ethanol and resuspended in hexane. For pegylation of the IONPs, 100 mg of different 3.5 kDa DPA-PEG linkers (Jenkem Tech USA) are dissolved in a mixture of CHCl₃ and HCON(CH₃)₂ (dimethylformamide (DMF)). The NP solution (20 mg Fe) is then added to the DPA-PEG solution and stirred for 4 hr at room temperature. Pegylated SFP NPs are precipitated overnight by addition of hexane and then resuspended in water. Trace amounts of aggregates are removed by high-speed centrifugation (20,000×g, 30 min), and the monodisperse SFP NPs are stored in water for further characterization and ncMHC conjugation. The concentration of iron in IONP products is determined by spectrophotometry at A410 in 2N HCL. Based on the molecular structure and diameter of SFP NPs (Fe₃O₄; 8+1 nm diameter) (Xie, J. et al. (2007) Adv Mater 19:3163; Xie, J. et al. (2006) Pure Appl. Chem. 78:1003-1014), Applicant estimates that SFP solutions containing 1 mg of iron contain 5×1014 NPs.

The nanoparticles can also be made by thermally decomposing or heating a nanoparticle precursor. In one embodiment, the nanoparticle is a metal or a metal oxide nanoparticle. In one embodiment, the nanoparticle is an iron oxide nanoparticle. In one embodiment, the nanoparticle is a gold nanoparticle. In one embodiment, provided herein are the nanoparticles prepared in accordance with the present technology. In one embodiment, provided herein is a method of making iron oxide nanoparticles comprising a thermal decomposition reaction of iron acetylacetonate. In one embodiment, the iron oxide nanoparticle obtained is water-soluble. In one aspect, the iron oxide nanoparticle is suitable for protein conjugation. In one embodiment, the method comprises a single-step thermal decomposition reaction.

TABLE 1
Functionalized PEG linkers

| Linker | Types of | PEG | M.W. | Functional | |
|---|---|---|---|---|---|
| Code | Nanoparticle | Linkers | (kDa) | group | Structure |
| A1 | Gold nanoparticle (GNP-C) | Thiol-PEG- carboxyl | 3.5 | Carboxyl (—COOH) | 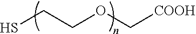 |
| A2 | Gold nanoparticle (GNP-N) | Thiol-PEG- amine | 3.5 | Amine (—NH₂) | 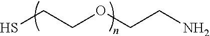 |
| S1 | Iron oxide Nanoparticle (SFP-C) | Dopamine- PEG- carboxyl | 3.5 | Carboxyl (—COOH) | 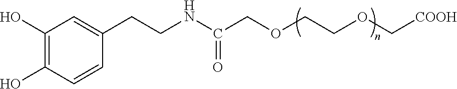 |
| S2 | Iron oxide Nanoparticle (SFP-N) | Dopamine- PEG-amine | 3.5 | Amine (—NH₂) | 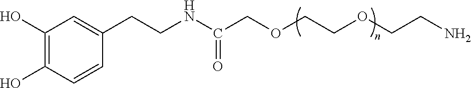 |
| S3 | Iron oxide Nanoparticle (SFP-Z) | Dopamine- PEG-azide | 3.5 | Azide (—N₃) | 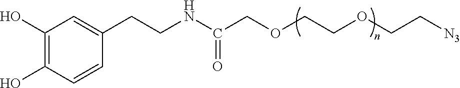 |
| S4 | Iron oxide Nanoparticle (SFP-M) | Dopamine- PEG- maleimide | 3.5 | 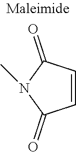 | 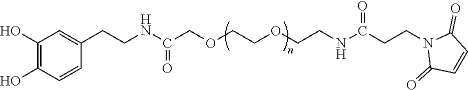 |
| S5 | Iron oxide Nanoparticle (SFP-O) | Dopamine- PEG- Orthopyridyl disulfide | 3.5 | 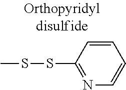 | 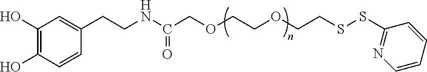 |
| P1 | Iron oxide Nanoparticle (PF-C) | carboxyl- PEG- carboxyl | 2.0 | Carboxyl (—COOH) | 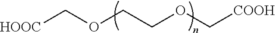 |
| P2 | Iron oxide Nanoparticle (PF-N) | Methoxy- PEG- amine | 2.0 | Amine (—NH₂) | 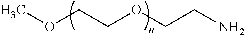 |
| P3 | Iron oxide Nanoparticle (PF-M) | Methoxy- PEG- maleimide | 2.0 | 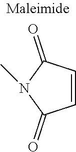 | 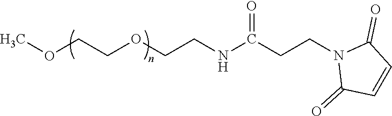 |
| P4 | Iron oxide Nanoparticle (PF-O) | Methoxy- PEG- Orthopyridyl disulfide | 2.0 | 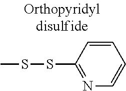 | 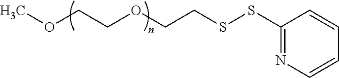 |
| P5 | Iron oxide Nanoparticle | PEG | 2.0 | Hydroxyl (—OH) | 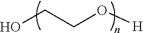 |
| | (PF) | | | | |

In one aspect, the thermal decomposition occurs in the presence of functionalized PEG molecules. Thermal decomposition to create soluble, stable nanoparticles with functionalized PEG linkers is described in Singha et al. “Peptide-MHC-based nanomedicines for autoimmunity function as T-cell receptor microclustering devices.” Nat Nanotechnol. 2017 July; 12 (7):701-710. Certain non-limiting examples of functionalized PEG linkers are shown in Table 1.

In one aspect, the thermal decomposition comprises heating an iron salt. In one aspect, the thermal decomposition comprises heating iron acetylacetonate. In one embodiment, the thermal decomposition comprises heating iron acetylacetonate in the presence of functionalized PEG molecules. In one embodiment, the thermal decomposition comprises heating iron acetylacetonate in the presence of benzyl ether and functionalized PEG molecules.

Without being bound by theory, in one embodiment, functionalized PEG molecules are used as reducing reagents and as surfactants. The method of making nanoparticles provided herein simplifies and improves conventional methods, which use surfactants that are difficult to be displaced, or are not displaced to completion, by PEG molecules to render the particles water-soluble. Conventionally, surfactants can be expensive (e.g., phospholipids) or toxic (e.g., Oleic acid or oleilamine). In another aspect, without being bound by theory, the method of making nanoparticles obviates the need to use conventional surfactants, thereby achieving a high degree of molecular purity and water solubility.

In one embodiment, the thermal decomposition involves iron acetylacetonate and benzyl ether and in the absence of conventional surfactants other than those employed herein.

In one embodiment, the temperature for the thermal decomposition is about 80° C. to about 300° C., or about 80° C. to about 200° C., or about 80° C. to about 150° C., or about 100° C. to about 250° C., or about 100° C. to about 200° C., or about 150° C. to about 250° C., or about 150° C. to about 250° C. In one embodiment, the thermal decomposition occurs within about 1 to about 2 hours of time.

In one embodiment, the method of making the iron oxide nanoparticles comprises a purification step, such as by using Miltenyi Biotec LS magnet column.

In one embodiment, the nanoparticles are stable at about 4° C. in phosphate buffered saline (PBS) without any detectable degradation or aggregation. In one embodiment, the nanoparticles are stable for at least 6 months.

In one aspect, provided herein is a method of making nanoparticle complexes comprising contacting ncMHC with iron oxide nanoparticles provided herein. Without being bound by theory, ncMHC encodes a Cysteine at its carboxyterminal end, which can react with the maleimide group in functionalized PEG at about pH 6.2 to about pH 6.5 for about 12 to about 14 hours.

In one aspect, the method of making nanoparticle complexes comprises a purification step, such as by using Miltenyi Biotec LS magnet column.

In certain aspects, ncMHC complex can be coupled to the nanoparticle core by one or more of covalently, non-covalently, or cross-linked, and optionally coupled through a linker. In certain aspects, the linker comprises polyethylene glycol or dextran. In certain aspects, the linker is polyethylene glycol or dextran. In further aspects, the linker may be less than 5 kD in size, and is optionally polyethylene glycol. In further aspects, the linker may be less than 5 kD in size, and is optionally dextran. In aspects involving a linker or linkers, the linkers may be the same or different from each other on a single nanoparticle core.

The term “linking molecule” or “linker” means a substance capable of linking with the substrate or particle and also capable of linking to an MHC complex.

In certain embodiments, the linker comprises polyethylene glycol with a molecular weight of less than 500 Daltons. In some embodiments, polyethylene glycol has a molecular weight of less than 1 kD, 2 kD, 3 kD, 4 kD, 5 kD, 6 kD, 7 kD, 8 kD, 9 kD, or 10 kD. In some embodiments, polyethylene glycol has a molecular weight up to and including 1 kD, 2 kD, 3 kD, 4 kD, 5 kD, 6 kD, 7 kD, 8 kD, 9 kD, or 10 kD. In some embodiments, polyethylene glycol has a molecular weight of between about 1 kD and about 5 kD, between about 2 kD and about 5 kD, or between about 3 kD and about 5 kD. In some embodiments, polyethylene glycol is functionalized with maleimide. In some embodiments, polyethylene glycol is functionalized with carboxyl group. In certain embodiments, the end of the linker that is in contact with the solid core is embedded in the solid core. In certain embodiments, the end of the linker that is in contact with the solid core is adsorbed to the solid core. In certain embodiments, the end of the linker that is in contact with the solid core forms a coordinated covalent bond with the solid core, especially with a metallic core (iron, iron oxide, or gold, etc.).

The coupling of a linker to a nanoparticle may be generated by chemically modifying the substrate or particle which typically involves the generation of “functional groups” on the surface, said functional groups being capable of binding to an ncMHC complex, and/or linking the optionally chemically modified surface of the surface or particle with covalently or non-covalently bound so-called “linking molecules,” followed by reacting the ncMHC or ncMHC complex with the particles obtained.

The term “functional groups” as used hereinbefore is not restricted to reactive chemical groups forming covalent bonds, but also includes chemical groups leading to an ionic interaction or hydrogen bonds with the MHC complex. Moreover, it should be noted that a strict distinction between “functional groups” generated at the surface and linking molecules bearing “functional groups” is not possible, since sometimes the modification of the surface requires the reaction of smaller linking molecules such as ethylene glycol with the particle surface.

The functional groups or the linking molecules bearing them may be selected from amino groups, carbonic acid groups, thiols, thioethers, disulfides, guanidino, hydroxyl groups, amine groups, vicinal diols, aldehydes, alpha-haloacetyl groups, mercury organyles, ester groups, acid halide, acid thioester, acid anhydride, isocyanates, isothiocyanates, sulfonic acid halides, imidoesters, diazoacetates, diazonium salts, 1,2-diketones, phosphonic acids, phosphoric acid esters, sulfonic acids, azolides, imidazoles, indoles, N-maleimides, alpha-beta-unsaturated carbonyl compounds, arylhalogenides or their derivatives.

Non-limiting examples for other linking molecules with higher molecular weights are nucleic acid molecules, polymers, copolymers, polymerizable coupling agents, silica, proteins, and chain-like molecules having a surface with the opposed polarity with respect to the substrate or particle. Nucleic acids can provide a link to affinity molecules containing themselves nucleic acid molecules, though with a complementary sequence with respect to the linking molecule.

In some embodiments, the linking molecule comprises polyethylene glycol. In some embodiments, the linking molecule comprises polyethylene glycol and maleimide. In some embodiments, the polyethylene glycol comprises one or more of a C₁-C₃ alkoxy group, —R¹⁰NHC(O)R—, —R¹⁰C(O)NHR—, —R¹⁰OC(O)R—, —R¹⁰C(O)OR—, wherein each R is independently H or C₁-C₆ alkyl and wherein each R¹⁰ is independently a bond or C₁-C₆ alkyl.

As examples for polymerizable coupling agents, diacetylene, styrene butadiene, vinylacetate, acrylate, acrylamide, vinyl compounds, styrene, silicone oxide, boron oxide, phosphorous oxide, borates, pyrrole, polypyrrole and phosphates can be cited.

ncMHC complexes can be coupled to nanoparticles by a variety of methods. One non-limiting example includes conjugation to NPs produced with PEG linkers carrying distal —NH₂ or —COOH groups that can be achieved via the formation of amide bonds in the presence of 1-Ethyl-3-[3-dimethylaminopropyl]carbodiimide hydrochloride (EDC). NPs with —COOH groups are first dissolved in 20 mM MES buffer, pH 5.5. N-hydroxysulfosuccinimide sodium salt (sulpha-NHS, Thermo scientific, Waltham, Mass., final concentration 10 mM) and EDC (Thermo scientific, Waltham, Mass., final concentration 1 mM) is added to the NP solution. After 20 min of stirring at room temperature, the NP solution is added drop-wise to the solution containing ncMHC monomers dissolved in 20 mM borate buffer (pH 8.2). The mixture is stirred for an additional 4 hr. To conjugate MHCs to NH2-functionalized NPs ncMHC complexes are first dissolved in 20 mM MES buffer, pH 5.5, containing 100 mM NaCl. Sulpha-NHS (10 mM) and EDC (5 mM) are then added to the MHC solution. The activated MHC molecules are then added to the NP solution in 20 mM borate buffer (pH 8.2), and stirred for 4 hr at room temperature.

To conjugate MHC to maleimide-functionalized NPs, ncMHC complexes are first incubated with Tributylphosphine (TBP, 1 mM) for 4 hr at room temperature. ncMHCs engineered to encode a free carboxyterminal Cys residue are then mixed with NPs in 40 mM phosphate buffer, pH 6.0, containing 2 mM EDTA, 150 mM NaCl, and incubated overnight at room temperature. MHCs of the ncMHC complexes are covalently bound with NPs via the formation of a carbon-sulfur bond between maleimide groups and the Cys residue.

Click chemistry can be used to conjugate ncMHC or avidin to NPs functionalized with azide groups. For this reaction, MHC or avidin molecules are first incubated with a suitable reagent comprising dibenzocyclooctyl (DBCO) functionality, for example: DBCO-NHS (Click Chemistry Tools, Scottdale, Ariz.) reagent for 2 hr at room temperature. Free DBCO-comprising molecules can be removed by dialysis overnight. MHC- or avidin-DBCO conjugates are then incubated with SFP-Z for 2 hr, resulting in formation of triazole bonds between ncMHCs or avidin molecules and NPs.

Unconjugated ncMHC complexes in the different ncMHC-NP conjugating reactions can be removed by extensive dialysis using methods known in the art. A non-limiting example is dialysis against PBS, pH 7.4, at 4° C. though 300 kDa molecular weight cut off membranes (Spectrum labs). Alternatively, ncMHC-conjugated IONPs can be purified by magnetic separation. The conjugated NPs can be concentrated by ultrafiltration through Amicon Ultra-15 units (100 kDa MWCO) and stored in PBS.

The surface of the substrate or particle can be chemically modified, for instance by the binding of phosphonic acid derivatives having functional reactive groups. One example of these phosphonic acid or phosphonic acid ester derivates is imino-bis(methylenphosphono) carbonic acid which can be synthesized according to the “Mannich-Moedritzer” reaction. This binding reaction can be performed with a substrate or a particle as directly obtained from the preparation process or after a pre-treatment (for instance with trimethylsilyl bromide). In the first case the phosphonic acid (ester) derivative may for instance displace components of the reaction medium which are still bound to the surface. This displacement can be enhanced at higher temperatures. Trimethylsilyl bromide, on the other hand, is believed to dealkylate alkyl group-containing phosphorous-based complexing agents, thereby creating new binding sites for the phosphonic acid (ester) derivative. The phosphonic acid (ester) derivative, or linking molecules bound thereto, may display the same functional groups as given above. A further example of the surface treatment of the substrate or particle involves heating in a diol such as ethylene glycol. It should be noted that this treatment may be redundant if the synthesis already proceeded in a diol. Under these circumstances the synthesis product directly obtained is likely to show the necessary functional groups. This treatment is, however, applicable to a substrate or a particle that was produced in N- or P-containing complexing agents. If such substrate or particle is subjected to an after-treatment with ethylene glycol, ingredients of the reaction medium (e.g. complexing agent) still binding to the surface can be replaced by the diol and/or can be dealkylated.

It is also possible to replace N-containing complexing agents still bound to the particle surface by primary amine derivatives having a second functional group. The surface of the substrate or particle can also be coated with silica. Silica allows a relatively simple chemical conjugation of organic molecules since silica easily reacts with organic linkers, such as triethoxysilane or chlorosilane. The particle surface may also be coated by homo- or copolymers. Examples for polymerizable coupling agents are: N-(3-aminopropyl)-3-mercaptobenzamidine, 3-(trimethoxysilyl)propylhydrazide and 3-trimethoxysilyl)propylmaleimide. Other non-limiting examples of polymerizable coupling agents are mentioned herein. These coupling agents can be used singly or in combination depending on the type of copolymer to be generated as a coating.

Another surface modification technique that can be used with substrates or particles containing oxidic transition metal compounds is conversion of the oxidic transition metal compounds by chlorine gas or organic chlorination agents to the corresponding oxychlorides. These oxychlorides are capable of reacting with nucleophiles, such as hydroxy or amino groups as often found in biomolecules. This technique allows generating a direct conjugation with proteins, for instance, via the amino group of lysine side chains. The conjugation with proteins after surface modification with oxychlorides can also be effected by using a bi-functional linker, such as maleimidopropionic acid hydrazide.

For non-covalent linking techniques, chain-type molecules having a polarity or charge opposite to that of the substrate or particle surface are particularly suitable. Examples for linking molecules which can be non-covalently linked to core/shell nanoparticles involve anionic, cationic or zwitter-ionic surfactants, acid or basic proteins, polyamines, polyamides, polysulfone or polycarboxylic acid. The hydrophobic interaction between substrate or particle and amphiphilic reagent having a functional reactive group can generate the necessary link. In particular, chain-type molecules with amphiphilic character, such as phospholipids or derivatized polysaccharides, which can be crosslinked with each other, are useful. The absorption of these molecules on the surface can be achieved by coincubation. The binding between affinity molecule and substrate or particle can also be based on non-covalent, self-organizing bonds. One example thereof involves simple detection probes with biotin as linking molecule and avidin- or streptavidin-coupled molecules.

Protocols for coupling reactions of functional groups to biological molecules can be found in the literature, for instance in “Bioconjugate Techniques” (Greg T. Hermanson, Academic Press 1996). The biological molecule (e.g., MHC molecule or derivative thereof) can be coupled to the linking molecule, covalently or non-covalently, in line with standard procedures of organic chemistry such as oxidation, halogenation, alkylation, acylation, addition, substitution or amidation. These methods for coupling the covalently or non-covalently bound linking molecule can be applied prior to the coupling of the linking molecule to the substrate or particle or thereafter. Further, it is possible, by means of incubation, to effect a direct binding of molecules to correspondingly pre-treated substrate or particles (for instance by trimethylsilyl bromide), which display a modified surface due to this pre-treatment (for instance a higher charge or polar surface).

### Methods of Triggering the Formation of Immunoregulatory Invariant NKT Cells

In certain embodiments, described herein, is a novel iNKT cell that expresses MAF, IL-10, IL-21 and at least one cell-surface marker selected from LAG3, CTLA4, SLAMF6, ITAG4 and combinations thereof. In certain embodiments, the cell is cultured or expanded in vitro. Such cells can be cultured or expanded in vitro by the ncMHC-sphingolipid nanoparticles described herein. Such culturing or expanding can be primed in vitro or after in vivo expansion in the body of an individual administered an ncMHC nanoparticle described herein. In certain embodiments, cultured or in vitro expanded iNKT cells can be administered to an individual in need thereof. In certain embodiments, the cells are administered to an individual that provided the NKT cells in an autologous manner.

The ncMHC-NPs disclosed herein are useful in methods of generating regulatory invariant NKT cells. In certain embodiments, the ncMHC-NPs are useful in methods of generating immunoregulatory iNKT cells in the liver of an individual. In certain embodiments, the ncMHC-NPs are useful in methods of generating immunoregulatory iNKT cells that express high levels of an immunoregulatory iNKT cell marker selected from: LAG3, CTLA4, SLAMF6, ITAG4, and combinations thereof. In certain embodiments, the ncMHC-NPs are useful in methods of generating immunoregulatory iNKT cells that express high levels of the immunoregulatory iNKT cell marker LAG3. In certain embodiments, the ncMHC-NPs are useful in methods of generating immunoregulatory iNKT cells that express high levels of the immunoregulatory iNKT cell marker CTLA4. In certain embodiments, the ncMHC-NPs are useful in methods of generating immunoregulatory iNKT cells that express high levels of the immunoregulatory iNKT cell marker SLAMF6. In certain embodiments, the ncMHC-NPs are useful in methods of generating immunoregulatory iNKT cells that express high levels of the immunoregulatory iNKT cell marker ITAG4. In certain embodiments, the ncMHC-NPs are useful in methods of generating immunoregulatory iNKT cells that express high levels of MAF. In certain embodiments, the ncMHC-NPs are useful in methods of generating immunoregulatory iNKT cells that express high levels of IL-10 and IL-21. High levels of expression can be determined using standard assays for RNA or protein detection. In certain embodiments, the non-classical MHC-NP does not comprise an immune activating co-stimulatory molecule or cytokine/cytokine receptor. In certain embodiments, the immunoregulatory invariant NKT cells are DC4+ invariant NKT cells. In various embodiments, the high levels of an immunoregulatory cell marker is in comparison to untreated, vehicle treated, or treatment with nanoparticles that lack CD1d molecules or a ceramide ligand bound to the CD1d molecules. In various embodiments, the high levels of an immunoregulatory cell marker is in comparison to treatment with αGalCer administered “free” without being bound to an ncMHC-NP. In certain embodiments, high-levels refer to an increase of 25%, 50%, 75%, 100%, or more compared to a control, e.g., untreated, vehicle treated, treatment with nanoparticles that lack CD1d molecules, or αGalCer administered without being bound to an ncMHC-NP. When referring to positivity of a marker, without qualification as high expression for said marker, it is recognized that all cells may express very low levels of a given cell-surface or intracellular marker, however, these cells are not considered to express the marker unless the levels are significant enough to return an appreciable result when compared to a control (e.g., sham treated or isotype control).

In certain embodiments, described herein, is a method of generating regulatory invariant NKT cells in an individual comprising administering to the individual in need thereof a non-classical MHC-nanoparticle complex comprising: a sphingolipid or an analog thereof; a non-classical MHC molecule; and a nanoparticle; wherein the sphingolipid or the analog thereof is associated with a binding groove of a non-classical MHC molecule, and wherein the non-classical MHC molecule is coupled to the nanoparticle. In certain embodiments, the administration is intravenous or subcutaneous. In certain embodiments, the administration is intravenous. In certain embodiments, the individual is afflicted with an autoimmune or inflammatory disease. In certain embodiments, the regulatory invariant NKT cells express high levels of marker selected from: LAG3, CTLA4, SLAMF6, ITAG4, and combinations thereof. In certain embodiments, the non-classical MHC-NP does not comprise an immune activating co-stimulatory molecule or cytokine/cytokine receptor. In certain embodiments, the regulatory invariant NKT cells are CD4+ invariant NKT cells.

In certain embodiments, described herein, is a method of generating regulatory invariant NKT cells in the liver of an individual comprising administering to the individual in need thereof a non-classical MHC-nanoparticle complex comprising: a sphingolipid or an analog thereof; a non-classical MHC molecule; and a nanoparticle; wherein the sphingolipid or the analog thereof is associated with a binding groove of a non-classical MHC molecule, and wherein the non-classical MHC molecule is coupled to the nanoparticle. In certain embodiments, the administration is intravenous or subcutaneous. In certain embodiments, the administration is intravenous. In certain embodiments, the individual is afflicted with a liver autoimmune or liver inflammatory condition. In certain embodiments, the liver autoimmune or liver inflammatory condition is selected from the list consisting of non-alcoholic fatty liver disease, non-alcoholic steatohepatitis, viral hepatitis, autoimmune hepatitis, primary biliary cholangitis, primary sclerosing cholangitis, cirrhosis, or a combination thereof. In certain embodiments, the regulatory invariant NKT cells express high levels of marker selected from: LAG3, CTLA4, SLAMF6, ITAG4, and combinations thereof. In certain embodiments, the non-classical MHC-NP does not comprise an immune activating co-stimulatory molecule or cytokine/cytokine receptor. In certain embodiments, the regulatory invariant NKT cells are CD4+ invariant NKT cells.

In certain embodiments, described herein, is a method of generating regulatory invariant NKT cells in an individual comprising administering to the individual in need thereof a non-classical MHC-nanoparticle complex comprising: a sphingolipid or an analog thereof; a non-classical MHC molecule; and a nanoparticle; wherein the sphingolipid or an analog thereof is associated with a binding groove of a non-classical MHC molecule, and wherein the non-classical MHC molecule is coupled to the nanoparticle. In certain embodiments, the administration is intravenous or subcutaneous. In certain embodiments, the administration is intravenous. In certain embodiments, the individual is afflicted with an autoimmune or inflammatory disease. In certain embodiments, the regulatory invariant NKT cells express high levels of IL-10, IL-21, or both IL-10 and IL-21. In certain embodiments, the non-classical MHC-NP does not comprise an immune activating co-stimulatory molecule or cytokine/cytokine receptor. In certain embodiments, the regulatory invariant NKT cells are CD4+ invariant NKT cells.

In certain embodiments, described herein, is a method of generating regulatory invariant NKT cells in the liver of an individual comprising administering to the individual in need thereof a non-classical MHC-nanoparticle complex comprising: a sphingolipid or an analog thereof; a non-classical MHC molecule; and a nanoparticle; wherein the sphingolipid or an analog thereof is associated with a binding groove of a non-classical MHC molecule, and wherein the non-classical MHC molecule is coupled to the nanoparticle. In certain embodiments, the administration is intravenous or subcutaneous. In certain embodiments, the administration is intravenous. In certain embodiments, the individual is afflicted with a liver autoimmune or liver inflammatory condition. In certain embodiments, the liver autoimmune or liver inflammatory condition is selected from the list consisting of non-alcoholic fatty liver disease, non-alcoholic steatohepatitis, viral hepatitis, autoimmune hepatitis, primary biliary cholangitis, primary sclerosing cholangitis, cirrhosis, or a combination thereof. In certain embodiments, the regulatory invariant NKT cells express high levels of IL-10, IL-21, or both IL-10 and IL-21. In certain embodiments, the non-classical MHC-NP does not comprise an immune activating co-stimulatory molecule or cytokine/cytokine receptor. In certain embodiments, the regulatory invariant NKT cells are CD4+ invariant NKT cells.

In certain embodiments, described herein, is a method of generating regulatory invariant NKT cells in an individual comprising administering to the individual in need thereof a non-classical MHC-nanoparticle complex comprising: a sphingolipid or an analog thereof; a non-classical MHC molecule; and a nanoparticle; wherein the sphingolipid or an analog thereof is associated with a binding groove of a non-classical MHC molecule, and wherein the non-classical MHC molecule is coupled to the nanoparticle. In certain embodiments, the administration is intravenous or subcutaneous. In certain embodiments, the administration is intravenous. In certain embodiments, the individual is afflicted with an autoimmune or inflammatory disease. In certain embodiments, the regulatory invariant NKT cells express MAF. In certain embodiments, the non-classical MHC-NP does not comprise an immune activating co-stimulatory molecule or cytokine/cytokine receptor. In certain embodiments, the regulatory invariant NKT cells are CD4+ invariant NKT cells.

In certain embodiments, described herein, is a method of generating regulatory invariant NKT cells in the liver of an individual comprising administering to the individual in need thereof a non-classical MHC-nanoparticle complex comprising: a sphingolipid or an analog thereof; a non-classical MHC molecule; and a nanoparticle; wherein the sphingolipid or an analog thereof is associated with a binding groove of a non-classical MHC molecule, and wherein the non-classical MHC molecule is coupled to the nanoparticle. In certain embodiments, the administration is intravenous or subcutaneous. In certain embodiments, the administration is intravenous. In certain embodiments, the individual is afflicted with a liver autoimmune or liver inflammatory condition. In certain embodiments, the liver autoimmune or liver inflammatory condition is selected from the list consisting of non-alcoholic fatty liver disease, non-alcoholic steatohepatitis, viral hepatitis, autoimmune hepatitis, primary biliary cholangitis, primary sclerosing cholangitis, cirrhosis, or a combination thereof. In certain embodiments, the regulatory invariant NKT cells express MAF. In certain embodiments, the non-classical MHC-NP does not comprise an immune activating co-stimulatory molecule or cytokine/cytokine receptor. In certain embodiments, the regulatory invariant NKT cells are CD4+ invariant NKT cells.

### Methods of Treating Autoimmune or Inflammatory Disorders

In another aspect, provided herein are methods of treating autoimmune or inflammatory disease using compositions comprising, consisting of, or consisting essentially of the ncMHC-NPs disclosed herein. In some embodiments, the disease is an autoimmune disease or disorder. In some embodiments, the disease is an inflammatory disease or disorder.

In some embodiments, the autoimmune or inflammatory disorder or disease may include, but is not limited to, Type I diabetes, transplantation rejection, multiple sclerosis, multiple-sclerosis related disorder, premature ovarian failure, scleroderma, Sjogren's disease/syndrome, lupus, vitiligo, alopecia (baldness), polyglandular failure, Grave's disease, hypothyroidism, polymyositis, pemphigus, Crohn's disease, colitis, autoimmune hepatitis, hypopituitarism, myocarditis, Addison's disease, autoimmune skin diseases, uveitis, pernicious anemia, hypoparathyroidism, rheumatoid arthritis, asthma, allergic asthma, autoimmune hepatitis, primary sclerosing cholangitis, primary biliary cholangitis, non-alcoholic steatohepatitis, cirrhosis, neuromyelitis optica spectrum disorder (Devic's disease, opticospinal multiple sclerosis (OSMS)), pemphigus vulgaris, inflammatory bowel disease (IBD), systemic lupus erythematosus (SLE), celiac disease, psoriasis, autoimmune cardiomyopathy, idiopathic dilated cardiomyopathy (IDCM), a myasthenia gravis, uveitis, ankylosing spondylitis, immune mediated myopathies, anti-phospholipid syndrome (ANCA+), atherosclerosis, dermatomyositis, chronic obstructive pulmonary disease (COPD), emphysema, spinal cord injury, ANCA-associated vasculitis, idiopathic pulmonary fibrosis, pulmonary hypertension, non-alcoholic fatty liver disease (NAFLD), non-alcoholic steatohepatitis (NASH) or psoriasis.

In some embodiments, the autoimmune or inflammatory disorder or disease is a liver autoimmune or inflammatory disease. In certain embodiments, the liver autoimmune or inflammatory disorder comprises autoimmune hepatitis, primary sclerosing cholangitis, primary biliary cirrhosis, non-alcoholic fatty liver disease (NAFLD), non-alcoholic steatohepatitis (NASH), or cirrhosis. In certain embodiments, the liver autoimmune or inflammatory disorder comprises autoimmune hepatitis, primary sclerosing cholangitis, or primary biliary cirrhosis.

Compositions of the disclosure may be conventionally administered parenterally, by injection, for example, intravenously, subcutaneously, intradermally, or intramuscularly. Additional formulations which are suitable for other modes of administration include oral formulations. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%. The preparation of an aqueous composition that contains an nc-MHC-nanoparticle complex that modifies the subject's immune condition will be known to those of skill in the art in light of the present disclosure. In certain embodiments, a composition may be inhaled (e.g., U.S. Pat. No. 6,651,655, which is specifically incorporated by reference in its entirety). In one embodiment, the ncMHC-nanoparticle complex is administered systemically. In specific embodiments, the ncMHC-NP complex or the compositions comprising a plurality of ncMHC-NP complexes can be administered intravenously.

Typically, compositions of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner. However, suitable dosage ranges are of the order of ten to several hundred nanograms or micrograms of sphingolipid ligand/ncMHC/nanoparticle complex per administration. Suitable regimes for initial administration and boosters are also variable, but are typified by an initial administration followed by subsequent administrations.

The manner of application may be varied widely. Any of the conventional methods for administration of a vaccine are applicable. These are believed to include oral application on a solid physiologically acceptable base or in a physiologically acceptable dispersion, parenterally, by injection and the like. The dosage of the sphingolipid ligand/ncMHC/nanoparticle complex will depend on the route of administration and will vary according to the size and health of the subject.

In many instances, it will be desirable to have multiple administrations of a sphingolipid ligand/ncMHC/nanoparticle complex, about, at least about, or at most about 3, 4, 5, 6, 7, 8, 9, 10 or more administrations. The administrations will normally range from 1, 2, 3, 4, 5, 6, or 7 day to twelve week intervals, more usually from one to two week intervals. Periodic boosters at intervals of every other day, twice a week, weekly, biweekly, monthly, or 0.1, 0.2, 0.3, 0.4, 0.5, 1, 2, 3, 4 or 5 years, usually two years, will be desirable to maintain the condition of the immune system. The course of the administrations may be followed by assays for autoreactive immune responses, cognate T_{R}1 cells, and T cell activity.

In certain aspects, a single dose of the ncMHC complex without including the nanoparticle core and any outer layer comprises about 0.001 mg/kg to about 2.0 mg/kg, or about 0.001 mg/kg to about 1.5 mg/kg, or about 0.001 mg/kg to about 1.4 mg/kg, or about 0.001 mg/kg to about 1.3 mg/kg, or about 0.001 mg/kg to about 1.2 mg/kg, or about 0.001 mg/kg to about 1.1 mg/kg, or about 0.001 mg/kg to about 1.0 mg/kg. In some embodiments, the single dose comprises from about 0.004 mg/kg to about 1.014 mg/kg, or from about 0.02 mg/kg to about 0.811 mg/kg, or from about 0.041 mg/kg to about 0.608 mg/kg, or from about 0.061 mg/kg to about 0.507 mg/kg, or from about 0.081 mg/kg to about 0.405 mg/kg, or from about 0.121 mg/kg to about 0.324 mg/kg, or from about 0.162 mg/kg to about 0.243 mg/kg. In some embodiments, the single dose comprises from about 0.004 mg/kg to about 1.015 mg/kg, or from about 0.004 mg/kg to about 1.0 mg/kg, or from about 0.004 mg/kg to about 0.9 mg/kg, or from about 0.004 mg/kg to about 0.8 mg/kg, or from about 0.004 mg/kg to about 0.7 mg/kg, or from about 0.004 mg/kg to about 0.6 mg/kg, or from about 0.004 mg/kg to about 0.5 mg/kg, or from about 0.004 mg/kg to about 0.4 mg/kg, or from about 0.004 mg/kg to about 0.3 mg/kg, or from about 0.004 mg/kg to about 0.2 mg/kg, or from about 0.004 mg/kg to about 0.1 mg/kg.

### Pharmaceutically Acceptable Excipients, Carriers, and Diluents

In some embodiments, the ncMHC-NPs are formulated into a pharmaceutical composition. Pharmaceutical compositions are formulated in a conventional manner using one or more pharmaceutically acceptable inactive ingredients that facilitate processing of the active agents into preparations that are used pharmaceutically. Proper formulation is dependent upon the route of administration chosen. A summary of pharmaceutical compositions, described herein, is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

Pharmaceutical compositions can also include surfactants, dispersing agents, and/or viscosity modulating agents. These agents include materials that can control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween ® 60 or 80, PEG, Tyloxapol, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K 15M, and HPMC K100M), carboxymethylcellulose sodium, ethylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide; and poloxamer 188); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 4000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizcers such as cellulose or triethyl cellulose can also be used as dispersing agents. In some cases, the pharmaceutical composition comprises a surfactant at between 0.01% and 0.5% (w/v). In some instances, the pharmaceutical composition comprises a surfactant at 0.01%, 0.05%, 0.1%, 0.2%, 0.4%, or 0.5% (w/v).

In certain embodiments, the ncMHC-NPs described herein are included in a pharmaceutical composition with a solubilizing, emulsifying, or dispersing agent. In certain embodiments, the solubilizing agent can allow high-concentration solutions of the ncMHC-NPs that exceed at least about 0.5 mg/mL, 1 mg/mL, 2 mg/mL, 5 mg/mL, 10 mg/mL, 15 mg/mL, or 20 mg/mL. Carbomers in an aqueous pharmaceutical composition serve as emulsifying agents and viscosity modifying agents. In certain embodiments, the pharmaceutically acceptable excipient comprises or consists of a carbomer. In certain embodiments, the carbomer comprises or consists of carbomer 910, carbomer 934, carbomer 934P, carbomer 940, carbomer 941, carbomer 1342, or combinations thereof. Cyclodextrins in an aqueous pharmaceutical composition serve as solubilizing and stabilizing agents. In certain embodiments, the pharmaceutically acceptable excipient comprises or consists of a cyclodextrin. In certain embodiments, the cyclodextrin comprises or consists of alpha cyclodextrin, beta cyclodextrin, gamma cyclodextrin, or combinations thereof. Lecithin in a pharmaceutical composition may serve as a solubilizing agent. In certain embodiments, the solubilizing agent comprises or consists of lecithin. Poloxamers in a pharmaceutical composition serve as emulsifying agents, solubilizing agents, and dispersing agents. In certain embodiments, the pharmaceutically acceptable excipient comprises or consists of a poloxamer. In certain embodiments, the poloxamer comprises or consists of poloxamer 124, poloxamer 188, poloxamer 237, poloxamer 338, poloxamer 407, or combinations thereof. Polyoxyethylene sorbitan fatty acid esters in a pharmaceutical composition serve as emulsifying agents, solubilizing agents, surfactants, and dispersing agents. In certain embodiments, the pharmaceutically acceptable excipient comprises or consists of a polyoxyethylene sorbitan fatty acid ester. In certain embodiments, the polyoxyethylene sorbitan fatty acid ester comprises or consists of polysorbate 20, polysorbate 21, polysorbate 40, polysorbate 60, polysorbate 61, polysorbate 65, polysorbate 80, polysorbate 81, polysorbate 85, polysorbate 120, or combinations thereof. Polyoxyethylene stearates in a pharmaceutical composition serve as emulsifying agents, solubilizing agents, surfactants, and dispersing agents. In certain embodiments, the pharmaceutically acceptable excipient comprises or consists of a polyoxyethylene stearate. In certain embodiments, the polyoxyethylene stearate comprises or consists of polyoxyl 2 stearate, polyoxyl 4 stearate, polyoxyl 6 stearate, polyoxyl 8 stearate, polyoxyl 12 stearate, polyoxyl 20 stearate, polyoxyl 30 stearate, polyoxyl 40 stearate, polyoxyl 50 stearate, polyoxyl 100 stearate, polyoxyl 150 stearate, polyoxyl 4 distearate, polyoxyl 8 distearate, polyoxyl 12 distearate, polyoxyl 32 distearate, polyoxyl 150 distearate, or combinations thereof. Sorbitan esters in a pharmaceutical composition serve as emulsifying agents, solubilizing agents, and non-ionic surfactants, and dispersing agents. In certain embodiments, the pharmaceutically acceptable excipient comprises or consists of a sorbitan ester. In certain embodiments, the sorbitan ester comprises or consists of sorbitan laurate, sorbitan oleate, sorbitan palmitate, sorbitan stearate, sorbitan trioleate, sorbitan sesquioleate, or combinations thereof. In certain embodiments, solubility can be achieved with a protein carrier. In certain embodiments the protein carrier comprises recombinant human albumin.

In certain embodiments, the non-classical MHC nanoparticles of the current disclosure are included in a pharmaceutical composition comprising one or more pharmaceutically acceptable excipients, carriers, and diluents. In certain embodiments, the non-classical MHC nanoparticles of the current disclosure are administered suspended in a sterile solution. In certain embodiments, the solution comprises about 0.9% NaCl. In certain embodiments, the solution comprises dextrose at about 5%. In certain embodiments, the solution further comprises one or more of: a buffer, for example, acetate, citrate, histidine, succinate, phosphate, bicarbonate and hydroxymethylaminomethane (Tris); a surfactant, for example, polysorbate 80 (Tween 80), polysorbate 20 (Tween 20), and poloxamer 188; a polyol/disaccharide/polysaccharide, for example, glucose, dextrose, mannose, mannitol, sorbitol, sucrose, trehalose, and dextran 40; an amino acid, for example, glycine or arginine; antioxidants, for example, ascorbic acid, methionine; or chelating agents, for example, EDTA or EGTA.

In certain embodiments, the non-classical MHC nanoparticles of the current disclosure are shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material.

The non-classical MHC nanoparticles when formulated, whether reconstituted or not, can be buffered at a certain pH, generally less than 8.0. In certain embodiments, the pH can be between 4.5 and 8.0, 4.5 and 7.5, 4.5 and 7.0, 4.5 and 6.5, 4.5 and 6.0, 4.5 and 5.5, 4.5 and 5.0. In certain embodiments, the pH can be between 5.0 and 6.0, 6.5, 7.0, 7.5, or 8.0. In certain embodiments, the pH can be between 6.0 and 6.5, 7.0, 7.5, or 8.0.

Also described herein are aqueous pharmaceutical compositions comprising the ncMHC and one or more pharmaceutically acceptable diluents, excipients, or carriers. In certain embodiments, the diluent, excipient, or carrier comprises a surfactant, solubilizing agent, or an emulsifier. In certain embodiments, the diluent, excipient, or carrier comprises a pH buffer. In certain embodiments, the concentration of the ncMHC in the aqueous composition is about 0.1 mg/mL to about 10 mg/mL. In certain embodiments, the concentration of the ncMHC in the aqueous composition is about 0.1 mg/mL to about 0.2 mg/mL, about 0.1 mg/mL to about 0.4 mg/mL, about 0.1 mg/mL to about 0.5 mg/mL, about 0.1 mg/mL to about 0.8 mg/mL, about 0.1 mg/mL to about 1 mg/mL, about 0.1 mg/mL to about 2 mg/mL, about 0.1 mg/mL to about 3 mg/mL, about 0.1 mg/mL to about 4 mg/mL, about 0.1 mg/mL to about 5 mg/mL, about 0.1 mg/mL to about 10 mg/mL, about 0.2 mg/mL to about 0.4 mg/mL, about 0.2 mg/mL to about 0.5 mg/mL, about 0.2 mg/mL to about 0.8 mg/mL, about 0.2 mg/mL to about 1 mg/mL, about 0.2 mg/mL to about 2 mg/mL, about 0.2 mg/mL to about 3 mg/mL, about 0.2 mg/mL to about 4 mg/mL, about 0.2 mg/mL to about 5 mg/mL, about 0.2 mg/mL to about 10 mg/mL, about 0.4 mg/mL to about 0.5 mg/mL, about 0.4 mg/mL to about 0.8 mg/mL, about 0.4 mg/mL to about 1 mg/mL, about 0.4 mg/mL to about 2 mg/mL, about 0.4 mg/mL to about 3 mg/mL, about 0.4 mg/mL to about 4 mg/mL, about 0.4 mg/mL to about 5 mg/mL, about 0.4 mg/mL to about 10 mg/mL, about 0.5 mg/mL to about 0.8 mg/mL, about 0.5 mg/mL to about 1 mg/mL, about 0.5 mg/mL to about 2 mg/mL, about 0.5 mg/mL to about 3 mg/mL, about 0.5 mg/mL to about 4 mg/mL, about 0.5 mg/mL to about 5 mg/mL, about 0.5 mg/mL to about 10 mg/mL, about 0.8 mg/mL to about 1 mg/mL, about 0.8 mg/mL to about 2 mg/mL, about 0.8 mg/mL to about 3 mg/mL, about 0.8 mg/mL to about 4 mg/mL, about 0.8 mg/mL to about 5 mg/mL, about 0.8 mg/mL to about 10 mg/mL, about 1 mg/mL to about 2 mg/mL, about 1 mg/mL to about 3 mg/mL, about 1 mg/mL to about 4 mg/mL, about 1 mg/mL to about 5 mg/mL, about 1 mg/mL to about 10 mg/mL, about 2 mg/mL to about 3 mg/mL, about 2 mg/mL to about 4 mg/mL, about 2 mg/mL to about 5 mg/mL, about 2 mg/mL to about 10 mg/mL, about 3 mg/mL to about 4 mg/mL, about 3 mg/mL to about 5 mg/mL, about 3 mg/mL to about 10 mg/mL, about 4 mg/mL to about 5 mg/mL, about 4 mg/mL to about 10 mg/mL, or about 5 mg/mL to about 10 mg/mL. In certain embodiments, the concentration of the ncMHC in the aqueous composition is about 0.1 mg/mL, about 0.2 mg/mL, about 0.4 mg/mL, about 0.5 mg/mL, about 0.8 mg/mL, about 1 mg/mL, about 2 mg/mL, about 3 mg/mL, about 4 mg/mL, about 5 mg/mL, or about 10 mg/mL. In certain embodiments, the concentration of the ncMHC in the aqueous composition is at least about 0.1 mg/mL, about 0.2 mg/mL, about 0.4 mg/mL, about 0.5 mg/mL, about 0.8 mg/mL, about 1 mg/mL, about 2 mg/mL, about 3 mg/mL, about 4 mg/mL, or about 5 mg/mL. In certain embodiments, the concentration of the ncMHC in the aqueous composition is at most about 0.2 mg/mL, about 0.4 mg/mL, about 0.5 mg/mL, about 0.8 mg/mL, about 1 mg/mL, about 2 mg/mL, about 3 mg/mL, about 4 mg/mL, about 5 mg/mL, or about 10 mg/mL.

Also described herein are kits comprising one or more of the non-classical MHC nanoparticles, described herein, in a suitable container and one or more additional components selected from: instructions for use; a diluent, an excipient, a carrier, and a device for administration.

In certain embodiments, described herein, is a method of preparing an autoimmune or inflammatory disease treatment comprising admixing one or more pharmaceutically acceptable excipients, carriers, or diluents and non-classical MHC nanoparticles the current disclosure. In certain embodiments, described herein, is a method of preparing an autoimmune disease or inflammatory disease treatment for storage or shipping comprising lyophilizing one or more non-classical MHC nanoparticles of the current disclosure.

Also described herein is a method of making a non-classical MHC-nanoparticle of the current disclosure comprising contacting a non-classical MHC molecule coupled to a nanoparticle to a sphingolipid or an analog thereof. In certain embodiments, the sphingolipid or an analog thereof comprises alpha-galactosylceramide. In certain embodiments, the non-classical MHC-NP does not comprise an immune activating co-stimulatory molecule or cytokine/cytokine receptor. In certain embodiments, the non-classical MHC-nanoparticle comprises a sphingolipid or an analog thereof. In certain embodiments, the sphingolipid or the analog thereof comprises a ceramide or an analog thereof. In certain embodiments, the ceramide or the analog thereof comprises alpha-galactosylceramide (KRN7000), alpha-C-galactosylceramide, alpha-glucuronosyl ceramide, beta-galactosylceramide, PBS-20, PBS-25, sulfatide, isoglobotriosylceramide (iGb3), or combinations thereof. In certain embodiments, the ceramide or an analog thereof comprises alpha-galactosylceramide (KRN7000). In certain embodiments, the non-classical MHC comprises CD1d. In certain embodiments, the CD1d is human CD1d. In certain embodiments, the CD1d comprises an amino acid residue sequence comprising at least about 90%, 95%, 97%, 98%, 99% identity to, or is identical to, any one of SEQ ID NOs: 1, 2, and 3. In certain embodiments, the CD1d comprises an amino acid residue sequence comprising at least about 90%, 95%, 97%, 98%, 99% identity to, or is identical to SEQ ID NO: 3. In certain embodiments, the CD1d comprises an amino acid residue sequence identical to SEQ ID NO: 3. In certain embodiments, the CD1d comprises an amino acid residue sequence comprising at least about 90%, 95%, 97%, 98%, 99% identity to, or is identical to SEQ ID NO: 4. In certain embodiments, the CD1d comprises an amino acid residue sequence identical to SEQ ID NO: 4. In certain embodiments, the non-classical MHC-nanoparticle comprises a β₂ microglobulin. In certain embodiments, the β₂ microglobulin comprises an amino acid residue sequence comprising at least about 90%, 95%, 97%, 98%, 99% identity to, or is identical to SEQ ID NO: 5. In certain embodiments, the β₂ microglobulin comprises an amino acid residue sequence identical to SEQ ID NO: 5. In certain embodiments, the nanoparticle comprises a metal, a metal oxide, a metal sulfide, a metal selenide, or a polymer. In certain embodiments, the metal or metal oxide comprises iron, iron oxide or gold. In certain embodiments, the metal or metal oxide comprises iron or iron oxide. In certain embodiments, the diameter of the nanoparticle is from about 1 nanometer to about 100 nanometers. In certain embodiments, the diameter is from about 5 nanometers to about 25 nanometers. In certain embodiments, the non-classical MHC molecule is covalently coupled to the nanoparticle. In certain embodiments, the non-classical MHC molecule is covalently coupled to the nanoparticle by a polymer linker. In certain embodiments, the polymer comprises dextran. In certain embodiments, the polymer linker is less than about 5 kilodaltons in size. In certain embodiments, the polymer linker comprises polyethylene glycol (PEG). In certain embodiments, the non-classical MHC molecule is coupled to the nanoparticle at a ratio of at least 10:1. In certain embodiments, the non-classical MHC molecule is coupled to the nanoparticle at a ratio of no more than about 1000:1. In certain embodiments, the non-classical MHC molecule is coupled to the nanoparticle at a ratio of no more than about 500:1. In certain embodiments, the non-classical MHC molecule is coupled to the nanoparticle at a ratio of no more than about 100:1.

### EXAMPLES

The following illustrative examples are representative of embodiments of compositions and methods described herein and are not meant to be limiting in any way.

### Example 1—Treatment of NOD.c3c4 Mice with αGalCer/CD1d-coated Nanoparticles Supresses Primary Biliary Cholangitis

The liver is the largest organ reservoir of iNKT cells. In liver inflammation, iNKT recruitment typically exacerbates tissue injury. αGalCer-induced activation of iNKT cells can induce liver damage. See Biburger, M. et al. (2005) “Alpha-galactosylceramide-induced liver injury in mice is mediated by TNF-alpha but independent of Kupffer cells.” J. Immunol. 175, 1540-1550. Additionally, microbial activation of iNKT cells can trigger liver autoimmunity. See Mattner, J. et al. (2005) “Exogenous and endogenous glycolipid antigens activate NKT cells during microbial infections.” Nature 434, 525-529. iNKT cells have also been shown to play a critical role in the pathogenesis of Concanavalin A-induced hepatitis (Takeda, K., et al. 2000 “Critical contribution of liver natural killer T cells to a murine model of hepatitis.” Proc. Natl. Acad. Sci. U.S.A. 97, 5498-5503); alcoholic hepatitis (Huang, W., et al., (2018) “The Role of CD1d and MR1 Restricted T Cells in the Liver.” Front. Immunol. 9, 2424); ischemia-reperfusion injury (Kuboki, S., et al. 2009 “Distinct contributions of CD4+ T cell subsets in hepatic ischemia/reperfusion injury.” Am. J. Physiol. Gastrointest. Liver Physiol. 296, G1054-1059); and drug-induced liver injuries (Kimura, K., et al. 2009 “Pathological role of CD44 on NKT cells in carbon tetrachloride-mediated liver injury.” Hepatol Res 39, 93-105). Furthermore, the frequency of liver iNKT cells in human Primary Biliary Cholangitis (PBC) is elevated, and introduction of a genetic iNKT cell deficiency into a murine model of PBC significantly decreased pathology. See Chuang, et al., 2008 “Natural killer T cells exacerbate liver injury in a transforming growth factor beta receptor II dominant-negative mouse model of primary biliary cirrhosis.” Hepatology 47, 571-580.

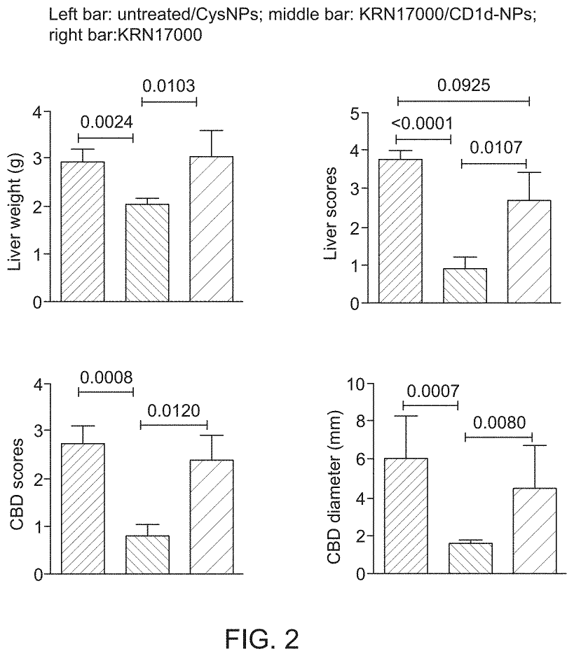

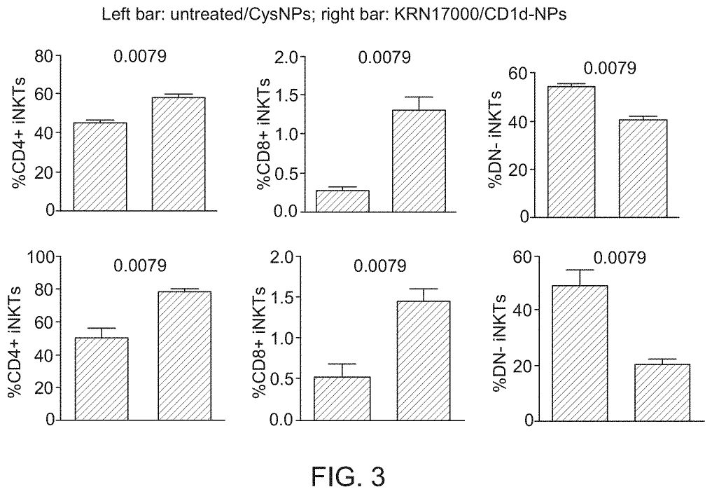

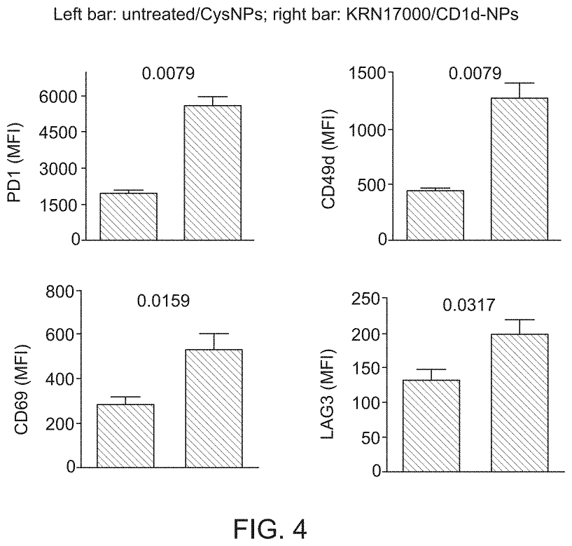

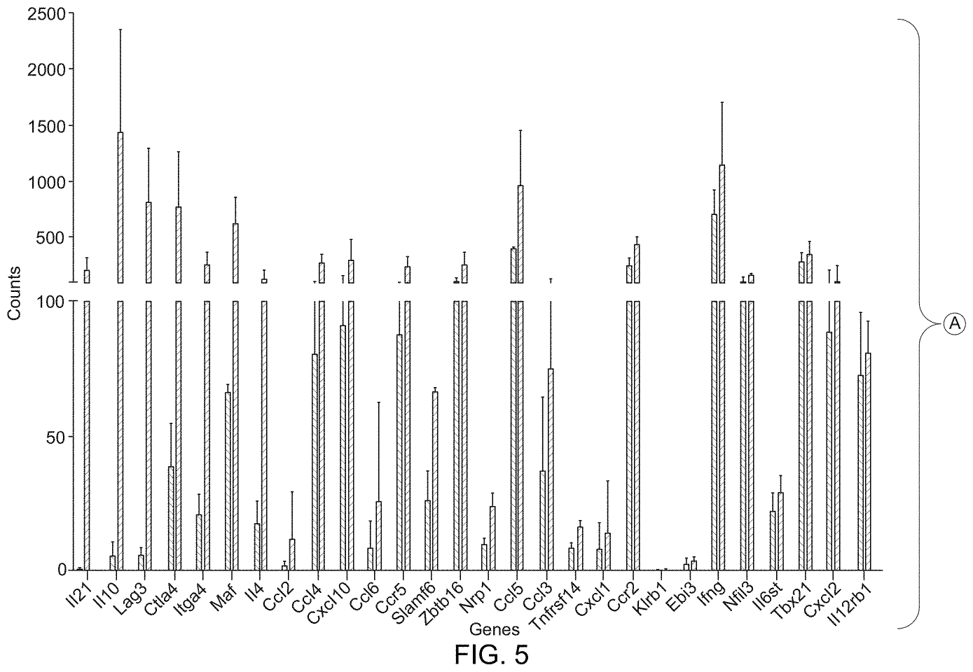

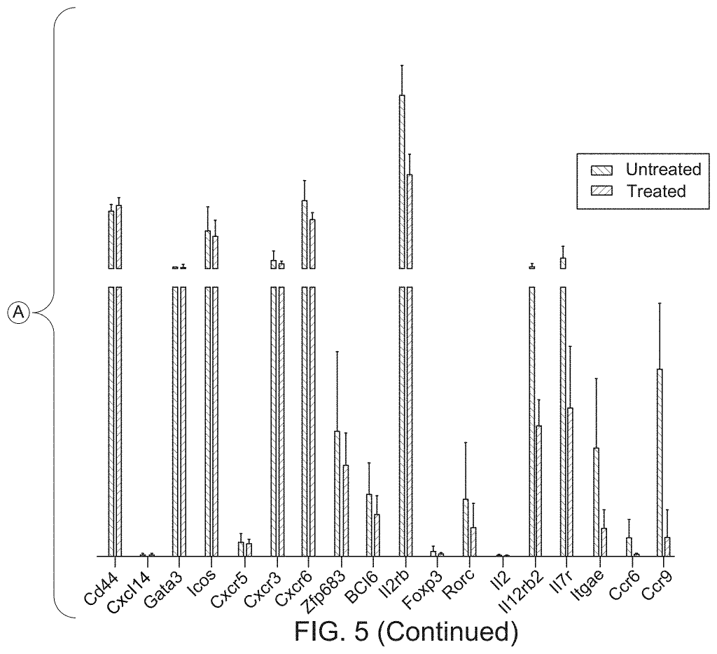

Surprisingly, treatment of NOD.c3c4 mice with αGalCer/CD1d-coated nanoparticles (NPs), but not αGalCer alone had a profound suppressive effect on spontaneous PBC-like disease in NOD.c3c4 mice as shown in FIG. 2. αGalCer/CD1d-NP treatment triggered a slight decrease in the iNKT content in spleen and lymph nodes (liver-draining and not), and a slight increase in the circulating frequency of these cells (not shown). The splenic and liver-associated iNKT cells from αGalCer/CD1d-NP-treated NOD.c3c4 mice were enriched for CD4+CD8− iNKTs, at the expense of CD4−CD8− iNKT cells as shown in FIG. 3; and expressed significantly higher levels of PD1, CD49d, CD69 and Lag-3, as compared to liver iNKT cells from control (untreated or Cys-NP-treated) mice as measured by flow cytometry as shown in FIG. 4.

### Example 2—Treatment of NOD.c3c4 Mice with αGalCer/CD1d-coated Nanoparticles Induces a Unique Set of iNKT Cells

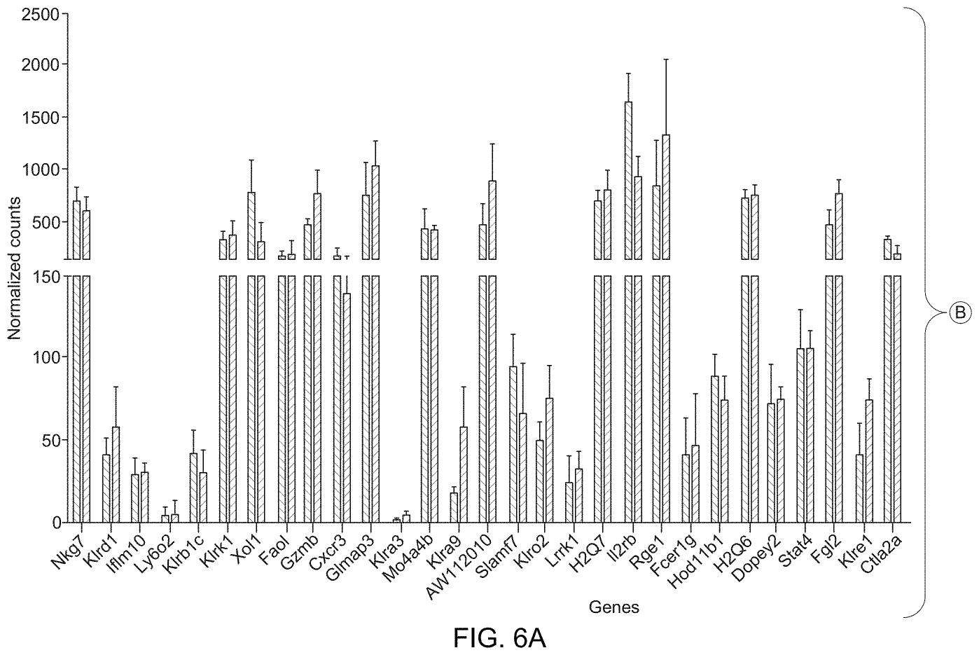

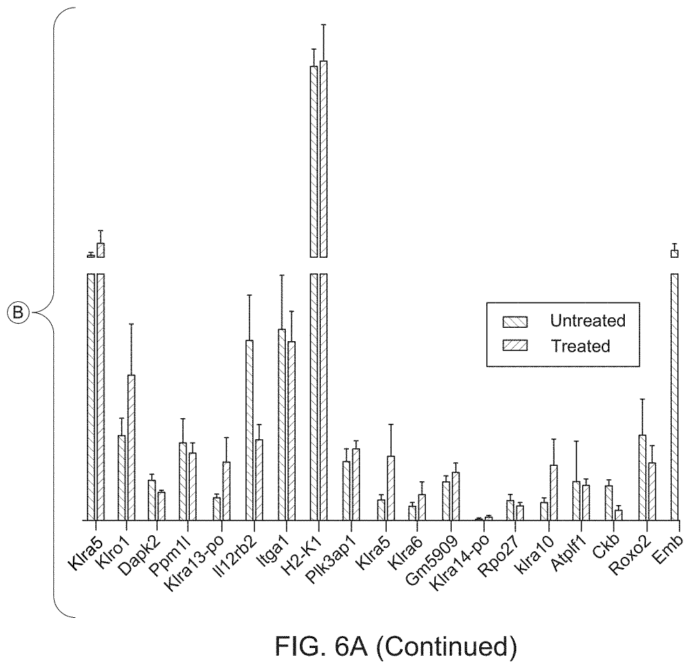

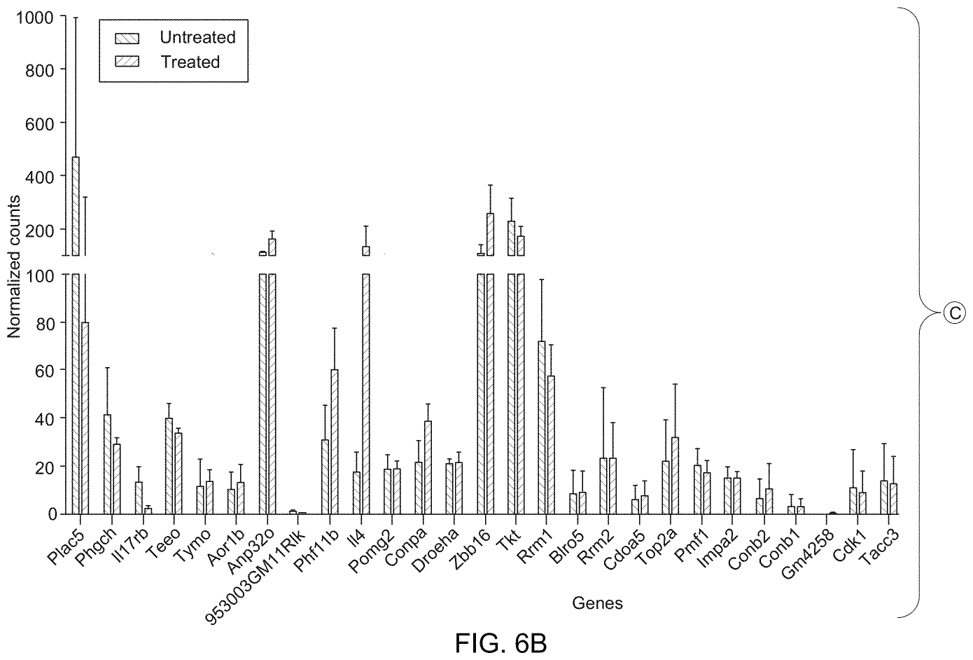

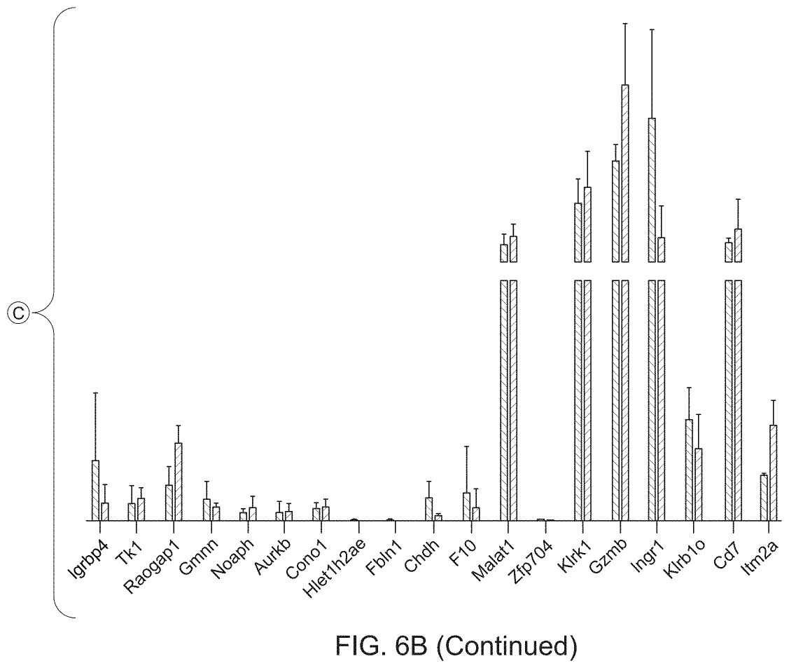

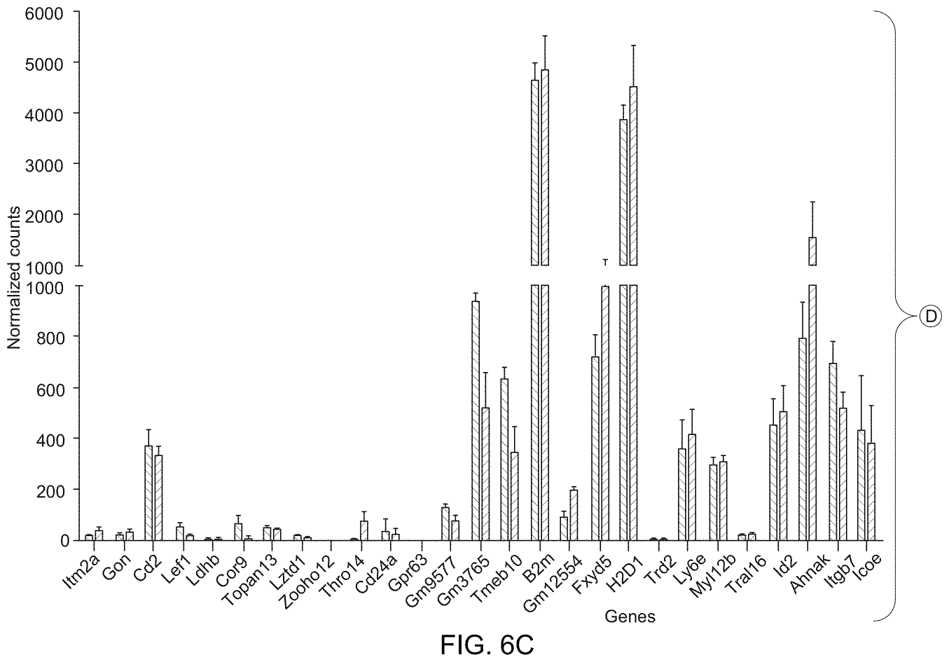

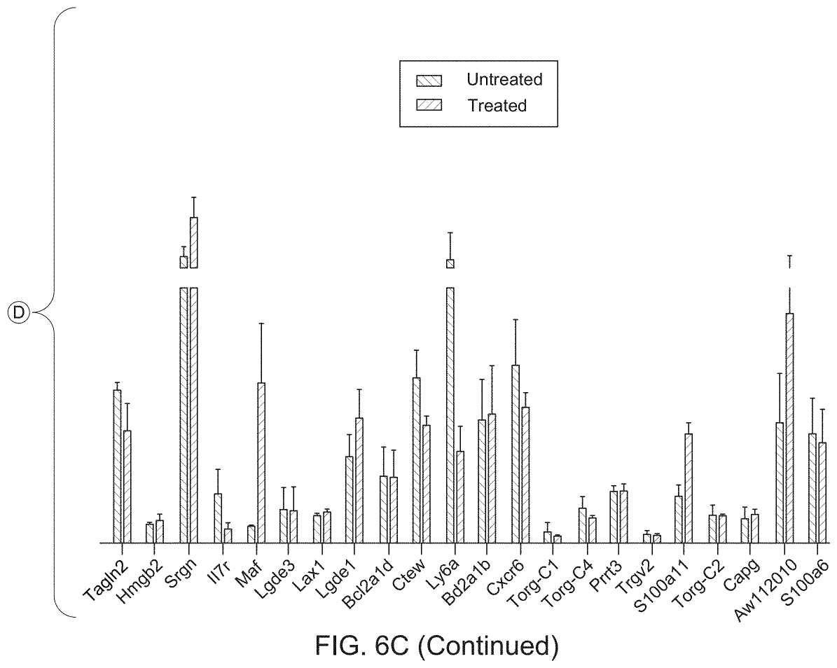

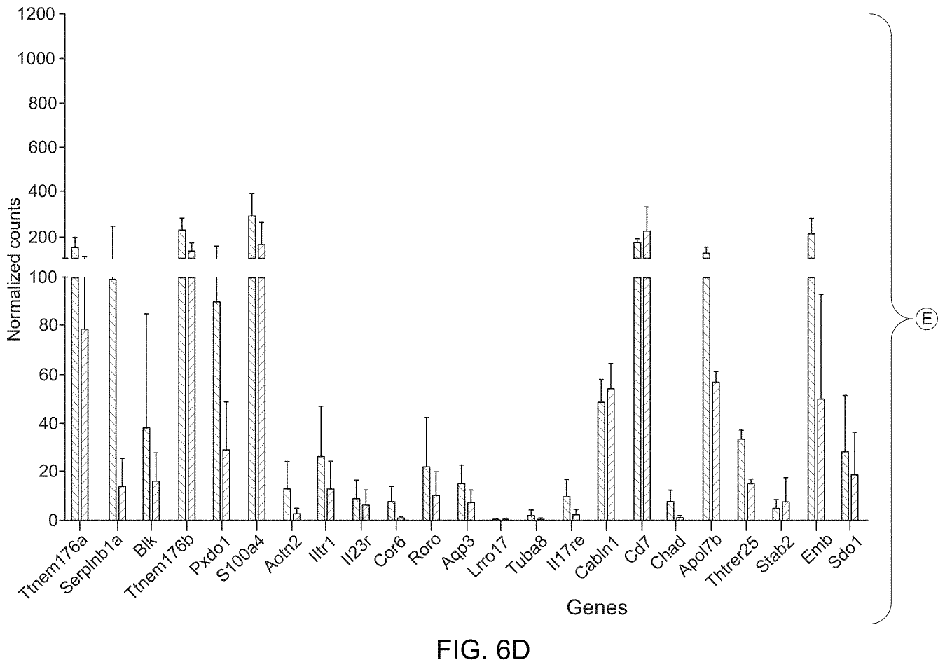

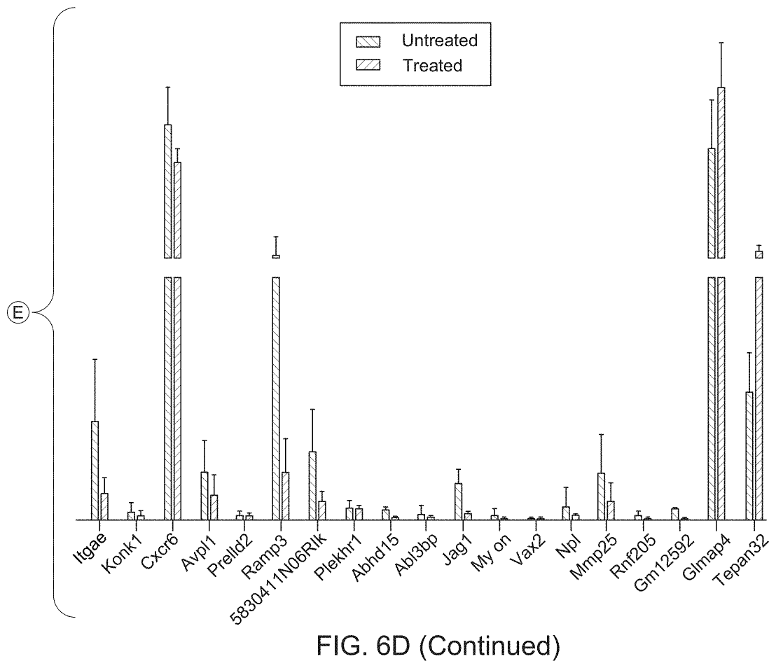

The above results suggested that αGalCer/CD1d-NP treatment might have triggered the formation of an immunoregulatory subset of iNKT cells. To investigate this, RNA sequencing (RNAseq) studies of liver iNKT cells sorted from αGalCer/CD1d-NP-treated vs. untreated NOD.c3c4 mice was performed. Indeed, these studies indicated that αGalCer/CD1d-NPs had re-programmed iNKT cells into a subset with high upregulation of the immunoregulatory cytokines IL-10 and IL-21 and the transcriptional regulator c-maf, in addition to various cell surface immunoregulatory molecules, such as Lag3, PD1, CTLA4 and TIGIT, among others. When considering genes known to be expressed in various iNKT cell subsets (Gapin, L. (2016). “Development of invariant natural killer T cells” Curr. Opin. Immunol. 39, 68-74) including iNKT1 (Zbtb16 (PLZF)-low, Tbx21 (Tbet)+, Gata3−, Rorc (RORgt)−, Foxp3−, Maf−, Cd24−, Cd44+, Klrb1 (NK1.1)+, Ifbg+, IL2−, Il4+, Il17−, Il10−, Il21−, Il2rb (Cd122)+, Mirlet7 (LET-7)+, Znf683 (HOBIT)-high), iNKT2 (Zbtb16-high, Tbx21−, Gata3+, Rorc−, Maf−, Foxp3−, Cd24−, Cd44+, Klrb1−, Ifng−, Il2−, Il4+, Il17−, Il10−, Il21−, Il17rb+, Tnfsf11 (RANKL)+; and iNKT17 (Zbtb16-int, Tbx21−, Gata3−, Rorc+, Foxp3−, Maf-low, Cd24−, Cd44+, Klrb1-low/−, Il2−, il4−, Ifng−, Il17+, Il10−, Il21−, Tgfbr2+, Tnfsf11+), the liver iNKT cells of non-treated NOD.c3c4 mice appeared to be rather unique (Zbtb16+, Tbx21+, Gata3+, Rorc-low, Foxp3−, Maf-low, Cd24-low, Cd44+, Klrb1+, Il2−, Il4-low, Ifng+, Il17−, Il10−, Il21−, Il2rb+, Il17rb-low, Tgfbr2+, Mirlet7−, Znf683−, Tnfsf11−). Remarkably, the liver iNKT cells from treated animals were somewhat similar to the latter, except that they were Rorc−, Maf-high, Il10-high, Il21-high, Il2rb+, Il17rb− and Tgfbr2-low. In addition, they expressed high levels of Lag3, Ctla4, Slamf6 and Itga4 (CD49d) (all negative in the liver iNKT cells of untreated mice), significantly downregulated Il7r, Itgae (CD103), Ccr6 and Tnfrsf14 (BTLA) and were Nrp1−. The liver iNKT cells of treated mice were also different from αGalCer-induced NKT10 cells, based on available gene expression data (Il10+, Klrb1-low, Nfil3+ (E4BP4, which regulates IL-10 and IL-13 expression), Nrp1+, Itga4+, Itgae−, Ctla4+, Fr4+, Bcl6−, Il7r+, Slamf6-high, Icos+) (Sag et al., 2014). Unlike these iNKT10 cells, the liver iNKT cells from treated mice were Nfli3−, Nrp1-low, Fr4−, Bcl6-low and Il7r-low. Likewise, they also appeared to be different from iNKT-FH cells (which express Icos) in that they express low levels of Bcl6 and lack expression of Cxcr5, or from lymph node FoxP3+ iNKT cells (Monteiro, M., et al. (2010). “Identification of regulatory Foxp3+ invariant NKT cells induced by TGF-beta” J. Immunol. 185, 2157-2163), adipose-tissue-resident FoxP3−Il 2+, Il10+, Zbtb16−Nfil3+ iNKT cells (Lynch, L., et al. (2015) “Regulatory iNKT cells lack expression of the transcription factor PLZF and control the homeostasis of T(reg) cells and macrophages in adipose tissue” Nat. Immunol. 16, 85-95) or the Breg-induced FoxP3− Zbtb16+ Nfil3− iNKT cells described recently (Oleinika, K., et al. (2018) “CD1d-dependent immune suppression mediated by regulatory B cells through modulations of iNKT cells” Nature communications 9, 684). These observations were substantiated by comparing the liver iNKT RNAseq data from αGalCer/CD1d-NP-treated and untreated mice to iNKT1, iNKT2, iNKT10 and iNKT17 RNAseq data from Engel et al. (Engel, I., et al. (2016). “Innate-like functions of natural killer T cell subsets result from highly divergent gene programs” Nat. Immunol. 17, 728-739.) (FIG. 6A to 6D)

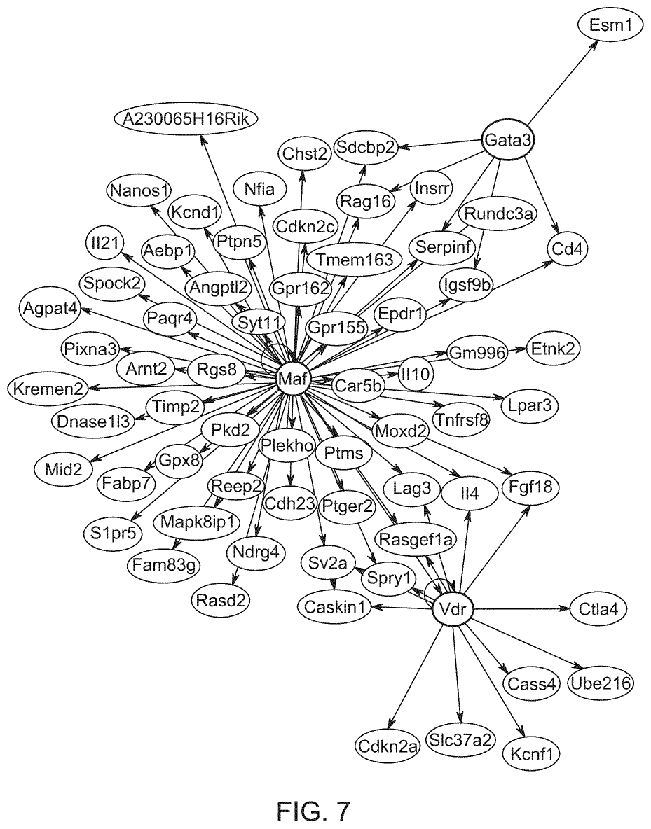

To ascertain if some of the gene expression differences that were observed between the liver iNKT cells of αGalCer/CD1d-NP-treated and untreated mice were driven, at least in part, by their unique transcriptional factor expression profile, which transcription factors were associated with expression of differentially expressed genes was determined. As shown in FIG. 7, 66 of the 135 upregulated protein-coding genes (and none of the 71 downregulated counterparts) were co-regulated by Maf, Gata3 and Vdr, three of the transcription factors that were significantly upregulated.

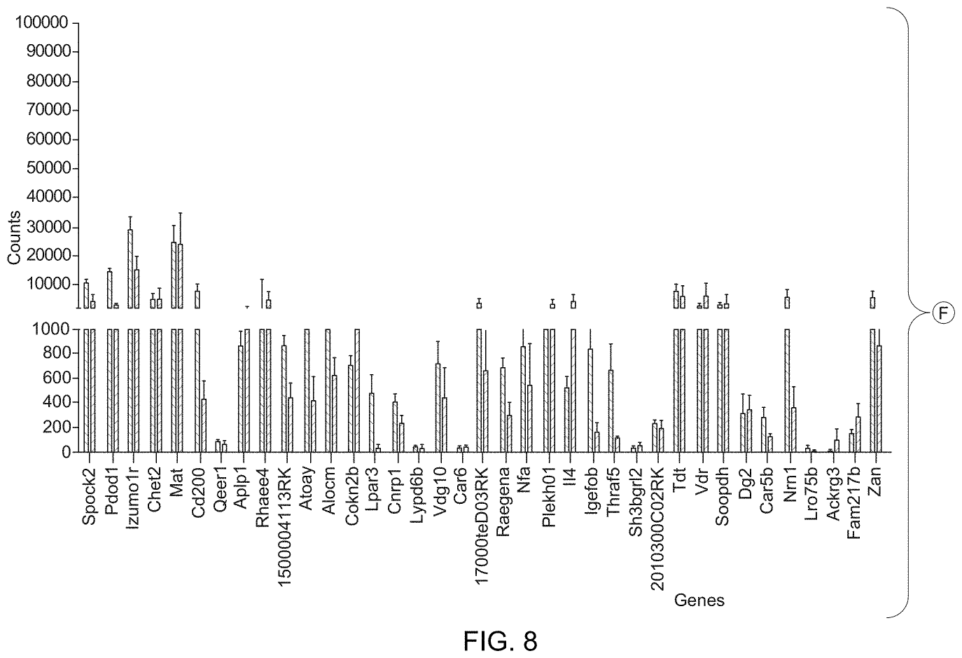

Interestingly, a number of these immunoregulatory molecules were also found to be upregulated in the TR1-like CD4+ T-cells that arise in NOD in response to pMHCII-NP therapy (Clemente-Casares et al., 2016). Interestingly, of the 238 differentially expressed genes in liver iNKT cells of αGalCer/CD1d-NP-treated vs. untreated mice, 138 (58%) were also found to be upregulated in the cognate TR1-like CD4+ T-cells of pMHCII-NP-treated mice as compared to conventional non-cognate CD4+ T-cells. Further comparison of the RNA-seq profiles of treatment-induced iNKT and TR1 cells revealed that 91 of the 238 genes differentially expressed in the liver iNKT cells of αGalCer/CD1d-NP-treated mice were shared by the TR1 cells. FIG. 8 shows the normalized gene counts for 66 of the genes with >4-fold differences in gene expression in liver of iNKT cells of treated vs. untreated mice, showing highly similar levels of expression. Consequently, these αGalCer/CD1d-NP-induced liver iNKT cells are called iNKTR1 cells.

### Example 3—Treatment of NOD.c3c4 Mice with αGalCer/CD1d-coated Nanoparticles Induce Regulatory iNKT Cells and Breg Cells

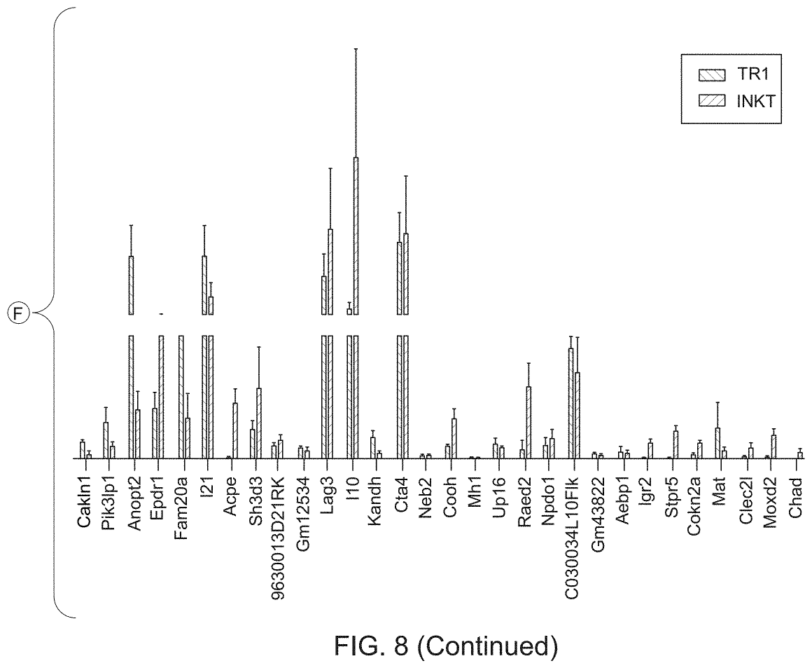

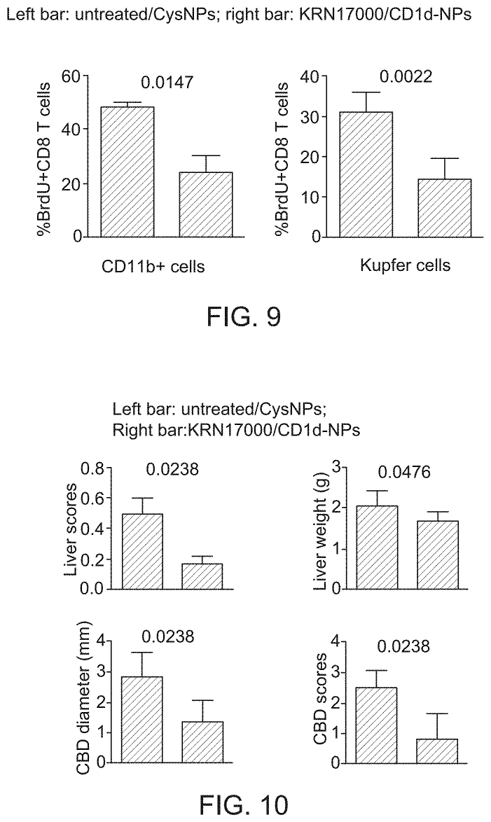

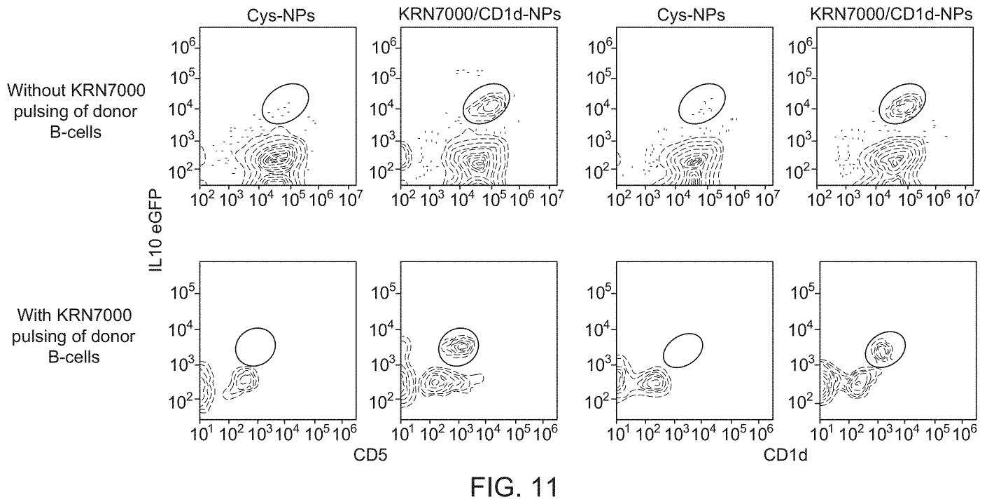

Next the mechanisms of disease suppression were investigated. The liver iNKT cells from αGalCer/CD1d-NP-treated mice could suppress the ability of CD11b+ APCs isolated form the liver-draining LNs and liver Kupffer cells from untreated mice, to present a model antigenic peptide (IGRP₂₀₆₋₂₁₄) to cognate TCR-transgenic CD8+ T-cells as shown in FIG. 9. In addition, the liver iNKT cells from αGalCer/CD1d-NP-treated NOD.c3c4 mice could transfer disease protection to NOD.c3c4.scid mice reconstituted with splenocytes from diseased NOD.c3c4 donors, as compared to liver iNKT cells from control NOD.c3c4 donors as shown in FIG. 10. Therapeutic activity in αGalCer7CD1d-NP-treated wild-type NOD.c3c4 mice could only be partially inhibited by IL-10 blockade and, to a lesser extent, IL-4 but not IFNγ blockade, suggesting that cytokines and/or mechanisms in addition to IL-10 were involved.

The above RNA expression data coupled to the transcriptional similarities with pMHCII-NP-induced TR1-like CD4+ T-cells and the cytokine/cytokine receptor blockade data suggested that the anti-inflammatory activity of αGalCer/CD1d-NPs involved the formation of a regulatory cell network arising downstream of the liver IL-21/IL-10-producing iNKT cells. To get further insights into downstream cellular targets, spinning-disk confocal intravital microscopy was used to track αGalCer/CD1d-NP-induced iNKT cells, Kuppfer cells, and B-lymphocytes in the livers of treated and untreated Ifng-Delta-ARE^{+/-} (NOD×B6) F1 mice, which also develop spontaneous PBC-like disease. To track iNKT cells in vivo, Cxcr6^{GFP/+} mice were used, in which 60-80% of liver eGFP+ cells are iNKT cells, the remainder being NK and T-cells (Geissmann et al., 2005; Liew et al., 2017). In sterile injury in the liver, recognition of endogenous glycolipid ligands on endothelial cells, Kupffer cells and other CD1d-expressing liver cells by iNKT cells result in the conversion of proinflammatory CCR2+ Ly6Chi monocytes into regulatory CX3CR1+ Ly6Clow monocytes, a process that is driven by iNKT-cell-derived IL-4. In Cxcr6^{GFP/+} Ifng-Delta-ARE^{+/-} (NOD×B6) F1 mice, bi-weekly treatment with αGalCer/CD1d-NPs triggered a massive increase in the number of liver iNKT cells as compared to untreated mice. Imaging of the livers of these mice 4 days after the last dose showed that these iNKT cells were randomly distributed throughout the liver and were highly motile, seeking and engaging rather static B-cells, labelled with a fluorochrome-labeled anti-CD19 mAb in vivo, but not Kupffer cells, labelled with F4/80-specific mAb. These interactions did not appear to result in B-cell killing. Although there were similar numbers of B-cells in the liver of untreated mice, they did engage in interactions with Cxcr6+ cells to the same extent. As expected, when imaged within 4 hours of an αGalCer/CD1d-NP dose, these iNKT cells stopped moving, consistent with an activated state. See Lee, W. Y., et al. (2010). “An intravascular immune response to Borrelia burgdorferi involves Kupffer cells and iNKT cells” Nat. Immunol. 11, 295-302.

The selective intra-hepatic iNKT-B-cell interaction was unexpected, as essentially all liver cells, including hepatocytes, liver sinusoidal endothelial cells, hepatocytes and hepatic stellate cells can present endogenous antigenic ligands to iNKT cells (Geissmann, F., et al. (2005) “Intravascular immune surveillance by CXCR6+ NKT cells patrolling liver sinusoids” PLoS Biol. 3, e113; and Lee et al. supra). It has been reported that a subset of follicular helper iNKT cells can provide cognate (by recognizing endogenous lipids in the context of CD1d on lipid antigen-specific B-cells) and non-cognate T-cell help to follicular B-cells (by promoting the activation of TFH cells recognizing cognate pMHCII on B-cells, also via CD1d-restricted interactions) and that marginal zone B-cells can trigger iNKT cell activation. It has also been shown that cognate iNKT-B-cell interactions can result in the differentiation of human Breg-like cells in vitro and trigger early increases in murine CD8− iNKT cells had this property. These observations, coupled to the intravital imaging observations and a role for CD1d-restricted interactions between αGalCer/CD1d-NP-induced iNKTR1 cells and B-cells, suggested the possibility that these iNKTR1 cells might be responsible for driving the differentiation of local B-cells into IL-10-producing Breg cells and that these B-cells might directly or indirectly contribute to therapeutic activity. It has previously been shown that TR1-like CD4+ T-cells elicited by pMHC class II-based nanomedicines trigger B-to-Breg differentiation not in a TGFα, IFNγ and IL-10-independent, but IL-21-dependent manner. See Clemente-Casares, X., et al. (2016) “Expanding antigen-specific regulatory networks to treat autoimmunity” Nature 530, 434-440.

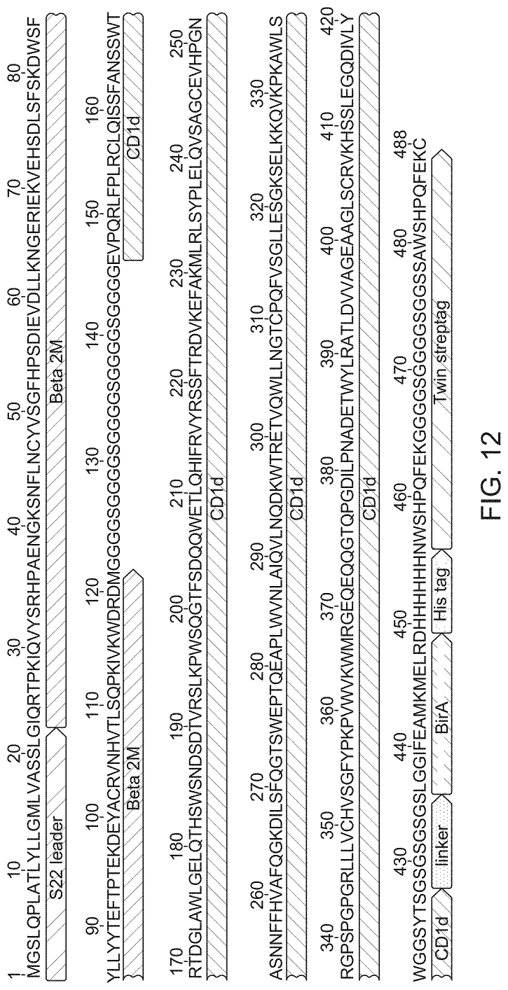

Since αGalCer/CD1d-NP-induced iNKTR1 cells express several hundred-fold higher levels of IL-21 and IL-10 mRNA than liver iNKT cells from untreated mice, and since IL-21R blockade abrogated the therapeutic activity of αGalCer/CD1d-NPs, whether iNKTR1 cells can drive Breg cell formation was investigated. This was done by transferring αGalCer-pulsed or non-pulsed B cells carrying an eGFP transgene in the Il10 locus into αGalCer/CD1d-NP-treated or untreated NOD.c3c4 mice, and enumerated the frequency of transferred CD5+ and CD1d^{high} B-cells that acquired eGFP expression within 7 days. Clearly, there was Breg cell formation in treated, but not untreated animals, and this occurred regardless of whether the B-cells were pulsed with αGalCer or not (FIG. 12). Thus, αGalCer/CD1d-NP treatment triggers iNKTR1 cell formation which then drive Breg cell formation by recognizing endogenous B-cell antigenic ligand(s) in a CD1d-restricted manner.

Collectively, these examples show that αGalCer/CD1d-coated NPs can readily trigger the differentiation of liver iNKT cells into a novel subset of regulatory iNKT cells, referred herein to iNKTR1 cells, that can dramatically suppress chronic liver inflammation in mouse models of PBC, and likely other liver inflammatory and autoimmune diseases as well. Thus, these compounds may offer a therapeutic solution of this and other chronic liver inflammatory diseases, and possibly inflammatory diseases elsewhere.

### Example 4—Treatment of Mice with αGalCer/CD1d-coated Nanoparticles Induces iNKT Cells with a Unique Phenotype

### Example 5—Loading of Cd1d Nanoparticles

Disclosed below is an example of a protocol for loading CD1d nanoparticles with αgalCer

### Reagents:

### 1. PBS (ETF)—pH 7.2-7.4

### 2. PBST(ETF) (0.5% Tween 20 in PBS)

### 3. CD1d-NPs

### KRN7000 (CAYMANCHEMICAL COMPANY, cat #11208)

### Protocol:

1. Bring the CD1d-NP particle solution to 37° C. before adding the lipid into the solution.
2. Make 2 L of boiling water (dd).
3. Calculate the amount of KRN7000 (CAYMANCHEMICAL COMPANY, cat #11208) required for the loading. Amount of KRN7000 required=(Total amount of CD1d (mg)/64)×Molar excess of KRN7000 to CD1d. As for example for 1 mg of CD1d the required KRN7000 (if we use 12:1 KRN7000 to CD1d in the reaction)=(1/64)×12=0.1875 mg. Consideration: MW of KRN7000 is 856 D and CD1d is 55 kD
4. Dissolve KRN7000 in 100% DMSO to a final concentration of 1 mg/mL.
5. Bring water bath to between 70-80° C. and sonicate for 5 minutes place tube comprising KRN7000/DMSO solution in water bath. (Note: This sonication step makes a monodisperse KRN7000 solution which aides effective loading of CD1d molecule).
8. Calculate the total vol. of reaction for lipid loading. For 0.255 mg of CD1d conjugated with NP particle, the final reaction vol. will be 2.5 mL of PBS and 0.05% Tween 20. Calculations: Considering the CD1d concentration in CD1d-NP solution is 1 mg/mL CD1d-NP-NP, 0.255 mL; PBST (0.5% Tween20), 0.25 mL; PBS, 1.948 mL; KRN solution, 0.047mL
9. Add KRN7000 solution to the nanoparticle solution.
10. Add the required amount of PBST and mix well.
11. Add the PBS to make the final volume as calculated.
12. Mix well and incubate at 37° C. for 2-3 hours, solution can be then stored at 4° C.
13. Purify the particles by magnetic column.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Sequences disclosed herein:

Human CD1d
SEQ ID NO: 1
MGCLLFLLLWALLQAWGSAEVPQRLFPLRCLQISSFANSSWTRTDGLAWLGELQTHSWSN
DSDTVRSLKPWSQGTFSDQQWETLQHIFRVYRSSFTRDVKEFAKMLRLSYPLELQVSAGC
EVHPGNASNNFFHVAFQGKDILSFQGTSWEPTQEAPLWVNLAIQVLNQDKWTRETVQWLL
NGTCPQFVSGLLESGKSELKKQVKPKAWLSRGPSPGPGRLLLVCHVSGFYPKPVWVKWMR
GEQEQQGTQPGDILPNADETWYLRATLDVVAGEAAGLSCRVKHSSLEGQDIVLYWGGSYT
SMGLIALAVLACLLFLLIVGFTSRFKRQTSYQGVL
Human CD1d signal sequence cleaved
SEQ ID NO: 2
EVPQRLFPLRCLQISSFANSSWTRTDGLAWLGELQTHSWSNDSDTVRSLKPWSQGTFSDQQ
WETLQHIFRVYRSSFTRDVKEFAKMLRLSYPLELQVSAGCEVHPGNASNNFFHVAFQGKDI
LSFQGTSWEPTQEAPLWVNLAIQVLNQDKWTRETVQWLLNGTCPQFVSGLLESGKSELKK
QVKPKAWLSRGPSPGPGRLLLVCHVSGFYPKPVWVKWMRGEQEQQGTQPGDILPNADET
WYLRATLDVVAGEAAGLSCRVKHSSLEGQDIVLYWGGSYTSMGLIALAVLACLLFLLIVGF
TSRFKRQTSYQGVL
Human CD1d signal sequence cleaved lacking TM and cytoplasmic
domain
SEQ ID NO: 3
EVPQRLFPLRCLQISSFANSSWTRTDGLAWLGELQTHSWSNDSDTVRSLKPWSQGTFSDQQ
WETLQHIFRVYRSSFTRDVKEFAKMLRLSYPLELQVSAGCEVHPGNASNNFFHVAFQGKDI
LSFQGTSWEPTQEAPLWVNLAIQVLNQDKWTRETVQWLLNGTCPQFVSGLLESGKSELKK
QVKPKAWLSRGPSPGPGRLLLVCHVSGFYPKPVWVKWMRGEQEQQGTQPGDILPNADET
WYLRATLDVVAGEAAGLSCRVKHSSLEGQDIVLYWGGSYTS
CD1d
SEQ ID NO: 4
EVPQRLFPLRCLQISSFANSSWTRTDGLAWLGELQTHSWSNDSDTVRSLKPWSQGTFSDQQ
WETLQHIFRVYRSSFTRDVKEFAKMLRLSYPLELQVSAGCEVHPGNASNNFFHVAFQGKDI
LSFQGTSWEPTQEAPLWVNLAIQVLNQDKWTRETVQWLLNGTCPQFVSGLLESGKSELKK
QVKPKAWLSRGPSPGPGRLLLVCHVSGFYPKPVWVKWMRGEQEQQGTQPGDILPNADET
WYLRATLDVVAGEAAGLSCRVKHSSLEGQDIVLYWGGSYT
b2M
SEQ ID NO: 5
IQRTPKIQVYSRHPAENGKSNFLNCYVSGFHPSDIEVDLLKNGERIEKVEHSDLSFSKDWSFY
LLYYTEFTPTEKDEYACRVNHVTLSQPKIVKWDRDM
single polypeptide encoding both CD1d and b2m
SEQ ID NO: 6
MGSLQPLATLYLLGMLVASSLGIQRTPKIQVYSRHPAENGKSNFLNCYVSGFHPSDIEVDLL
KNGERIEKVEHSDLSFSKDWSFYLLYYTEFTPTEKDEYACRVNHVTLSQPKIVKWDRDMGG
GGSGGGGSGGGGSGGGGSGGGGEVPQRLFPLRCLQISSFANSSWTRTDGLAWLGELQTHS
WSNDSDTVRSLKPWSQGTFSDQQWETLQHIFRVYRSSFTRDVKEFAKMLRLSYPLELQVSA
GCEVHPGNASNNFFHVAFQGKDILSFQGTSWEPTQEAPLWVNLAIQVLNQDKWTRETVQW
LLNGTCPQFVSGLLESGKSELKKQVKPKAWLSRGPSPGPGRLLLVCHVSGFYPKPVWVKW
MRGEQEQQGTQPGDILPNADETWYLRATLDVVAGEAAGLSCRVKHSSLEGQDIVLYWGGS
YTSGSGSGSGSLGGIFEAMKMELRDHHHHHHNWSHPQFEKGGGGSGGGGSGGSSAWSHP
QFEKC
nucleic acid sequence encoding-single polypeptide encoding both
CD1d and b2m
SEQ ID NO: 7
ATGGGTTCTCTGCAACCGCTGGCCACCTTGTACCTGCTGGGTATGCTGGTCGCTAGCAG
CCTCGGAATCCAGCGTACTCCAAAGATTCAGGTTTACTCACGTCATCCAGCAGAGAATG
GAAAGTCAAATTTCCTGAATTGCTATGTGTCTGGGTTTCATCCATCCGACATTGAAGTTG
ACTTACTGAAGAATGGAGAGAGAATTGAAAAAGTGGAGCATTCAGACTTGTCTTTCAGC
AAGGACTGGTCTTTCTATCTCTTGTACTACACTGAATTCACCCCCACTGAAAAAGATGA
GTATGCCTGCCGTGTGAACCATGTGACTTTGTCACAGCCCAAGATAGTTAAGTGGGATC
GAGACATGGGTGGCGGAGGGTCTGGAGGAGGGGGATCTGGAGGTGGAGGCTCCGGAG
GAGGTGGATCAGGAGGAGGTGGAGAAGTCCCGCAAAGGCTTTTCCCCCTCCGCTGCCTC
CAGATCTCGTCCTTCGCCAATAGCAGCTGGACGCGCACCGACGGCTTGGCGTGGCTGGG
GGAGCTGCAGACGCACAGCTGGAGCAACGACTCGGACACCGTCCGCTCTCTGAAGCCT
TGGTCCCAGGGCACGTTCAGCGACCAGCAGTGGGAGACGCTGCAGCATATATTTCGGGT
TTATCGAAGCAGCTTCACCAGGGACGTGAAGGAATTCGCCAAAATGCTACGCTTATCCT
ATCCCTTGGAGCTCCAGGTGTCCGCTGGCTGTGAGGTGCACCCTGGGAACGCCTCAAAT
AACTTCTTCCATGTAGCATTTCAAGGAAAAGATATCCTGAGTTTCCAAGGAACTTCTTG
GGAGCCAACCCAAGAGGCCCCACTTTGGGTAAACTTGGCCATTCAAGTGCTCAACCAG
GACAAGTGGACGAGGGAAACAGTGCAGTGGCTCCTTAATGGCACCTGCCCCCAATTTGT
CAGTGGCCTCCTTGAGTCAGGGAAGTCGGAACTGAAGAAGCAAGTGAAGCCCAAGGCC
TGGCTGTCCCGTGGCCCCAGTCCTGGCCCTGGCCGTCTGCTGCTGGTGTGCCATGTCTCA
GGATTCTACCCAAAGCCTGTATGGGTGAAGTGGATGCGGGGTGAGCAGGAGCAGCAGG
GCACTCAGCCAGGGGACATCCTGCCCAATGCTGACGAGACATGGTATCTCCGAGCAAC
CCTGGATGTGGTGGCTGGGGAGGCAGCTGGCCTGTCCTGTCGGGTGAAGCACAGCAGTC
TAGAGGGCCAGGACATCGTCCTCTACTGGGGTGGGAGCTACACCTCCGGTAGTGGTAGT
GGTAGTGGATCTCTGGGTGGTATCTTCGAGGCTATGAAGATGGAGCTGCGCGATCATCA
CCATCACCATCACAACTGGAGCCACCCTCAGTTCGAGAAGGGAGGTGGAGGCTCAGGA
GGTGGAGGCTCTGGAGGCTCTAGTGCATGGAGCCACCCTCAGTTCGAGAAGTGTTGA

1. A non-classical MHC-nanoparticle comprising:
a) a sphingolipid or an analog thereof;
b) a non-classical MHC molecule; and
c) a nanoparticle;
wherein the sphingolipid or the analog thereof is associated with a binding groove of the non-classical MHC molecule, and wherein the non-classical MHC molecule is coupled to the nanoparticle.

2. The non-classical MHC-nanoparticle of claim 1, wherein the non-classical MHC-nanoparticle does not comprise an immune activating co-stimulatory molecule or cytokine/cytokine receptor.

3. The non-classical MHC-nanoparticle of claim 1, wherein the non-classical MHC-nanoparticle comprises a sphingolipid or an analog thereof.

4. The non-classical MHC-nanoparticle claim 3, wherein the sphingolipid or the analog thereof comprises a ceramide or an analog thereof.

5. The non-classical MHC-nanoparticle of claim 4, wherein the ceramide or the analog thereof comprises alpha-galactosylceramide (KRN7000), alpha-C-galactosylceramide, alpha-glucuronosyl ceramide, beta-galactosylceramide, PBS-20, PBS-25, sulfatide, isoglobotriosylceramide (iGb3), or combinations thereof.

6. The non-classical MHC-nanoparticle of claim 5, wherein the ceramide or the analog thereof comprises alpha-galactosylceramide (KRN7000).

7. The non-classical MHC-nanoparticle of claim 1, wherein the non-classical MHC comprises CD1d.

8. The non-classical MHC-nanoparticle of claim 7, wherein the CD1d is human CD1d.

9. The non-classical MHC-nanoparticle of claim 8, wherein the CD1d comprises an amino acid residue sequence comprising at least about 90%, 95%, 97%, 98%, 99% identity to, or is identical to, any one of SEQ ID NOs: 1, 2, 3 or 4.

10. The non-classical MHC-nanoparticle of claim 9, wherein the CD1d comprises an amino acid residue sequence comprising at least about 90%, 95%, 97%, 98%, 99% identity to, or is identical to SEQ ID NO: 3.

11. (canceled)

12. The non-classical MHC-nanoparticle of claim 9, wherein the CD1d comprises an amino acid residue sequence comprising at least about 90%, 95%, 97%, 98%, 99% identity to, or is identical to SEQ ID NO: 4.

13. (canceled)

14. The non-classical MHC-nanoparticle of claim 1, wherein the non-classical MHC-nanoparticle comprises a β2 microglobulin.

15. The non-classical MHC-nanoparticle of claim 14, wherein the β2 microglobulin comprises an amino acid residue sequence comprising at least about 90%, 95%, 97%, 98%, 99% identity to, or is identical to SEQ ID NO: 5.

16. (canceled)

17. The non-classical MHC-nanoparticle of claim 1, wherein the non-classical MHC-nanoparticle comprises a metal, a metal oxide, a metal sulfide, a metal selenide, or a polymer.

18. The non-classical MHC-nanoparticle of claim 14, wherein the metal or metal oxide comprises iron, iron oxide or gold.

19. (canceled)

20. The non-classical MHC-nanoparticle of claim 1, wherein the diameter of the nanoparticle is from about 1 nanometer to about 100 nanometers.

21. The non-classical MHC-nanoparticle of claim 20, wherein the diameter is from about 5 nanometers to about 25 nanometers.

22. The non-classical MHC-nanoparticle of claim 1, wherein the non-classical MHC molecule is covalently coupled to the nanoparticle.

23. The non-classical MHC-nanoparticle of claim 1, wherein the non-classical MHC molecule is covalently coupled to the nanoparticle by a polymer linker.

24. The non-classical MHC-nanoparticle of claim 23, wherein the polymer linker comprises dextran or polyethylene glycol (PEG).

25. The non-classical MHC-nanoparticle of claim 23, wherein the polymer linker is less than about 5 kilodaltons in size.

26. (canceled)

27. The non-classical MHC-nanoparticle of claim 1, wherein the non-classical MHC molecule is coupled to the nanoparticle at a ratio of at least 10:1; or
wherein the non-classical MHC molecule is coupled to the nanoparticle at a ratio of no more than about 1000:1; or
wherein the non-classical MHC molecule is coupled to the nanoparticle at a ratio of no more than about 500:1; or
wherein the non-classical MHC molecule is coupled to the nanoparticle at a ratio of no more than about 100:1.

28-30. (canceled)

31. A composition comprising a plurality of the non-classical MHC-nanoparticle of claim 1 and a pharmaceutically acceptable excipient, diluent, or carrier.

32. The composition of claim 31, formulated for intravenous or subcutaneous administration.

33-42. (canceled)

43. A method of treating an autoimmune or inflammatory disorder in an individual comprising administering to the individual the non-classical MHC-nanoparticle of claim 1, thereby treating the individual with the autoimmune or inflammatory disorder.

44-54. (canceled)


SEQUENCE LISTING (7 sequences)

SEQ ID NO: 1 - protein, 335 aa, Homo sapiens
MGCLLFLLLWALLQAWGSAEVPQRLFPLRCLQISSFANSSWTRTDGLAWLGELQTHSWSNDSDTVRSLKPWSQGTFSDQQWETLQHIFRVYRSSFTRDVKEFAKMLRLSYPLELQVSAGCEVHPGNASNNFFHVAFQGKDILSFQGTSWEPTQEAPLWVNLAIQVLNQDKWTRETVQWLLNGTCPQFVSGLLESGKSELKKQVKPKAWLSRGPSPGPGRLLLVCHVSGFYPKPVWVKWMRGEQEQQGTQPGDILPNADETWYLRATLDVVAGEAAGLSCRVKHSSLEGQDIVLYWGGSYTSMGLIALAVLACLLFLLIVGFTSRFKRQTSYQGVL

SEQ ID NO: 2 - protein, 316 aa, Homo sapiens
EVPQRLFPLRCLQISSFANSSWTRTDGLAWLGELQTHSWSNDSDTVRSLKPWSQGTFSDQQWETLQHIFRVYRSSFTRDVKEFAKMLRLSYPLELQVSAGCEVHPGNASNNFFHVAFQGKDILSFQGTSWEPTQEAPLWVNLAIQVLNQDKWTRETVQWLLNGTCPQFVSGLLESGKSELKKQVKPKAWLSRGPSPGPGRLLLVCHVSGFYPKPVWVKWMRGEQEQQGTQPGDILPNADETWYLRATLDVVAGEAAGLSCRVKHSSLEGQDIVLYWGGSYTSMGLIALAVLACLLFLLIVGFTSRFKRQTSYQGVL

SEQ ID NO: 3 - protein, 282 aa, Homo sapiens
EVPQRLFPLRCLQISSFANSSWTRTDGLAWLGELQTHSWSNDSDTVRSLKPWSQGTFSDQQWETLQHIFRVYRSSFTRDVKEFAKMLRLSYPLELQVSAGCEVHPGNASNNFFHVAFQGKDILSFQGTSWEPTQEAPLWVNLAIQVLNQDKWTRETVQWLLNGTCPQFVSGLLESGKSELKKQVKPKAWLSRGPSPGPGRLLLVCHVSGFYPKPVWVKWMRGEQEQQGTQPGDILPNADETWYLRATLDVVAGEAAGLSCRVKHSSLEGQDIVLYWGGSYTS

SEQ ID NO: 4 - protein, 281 aa, Homo sapiens
EVPQRLFPLRCLQISSFANSSWTRTDGLAWLGELQTHSWSNDSDTVRSLKPWSQGTFSDQQWETLQHIFRVYRSSFTRDVKEFAKMLRLSYPLELQVSAGCEVHPGNASNNFFHVAFQGKDILSFQGTSWEPTQEAPLWVNLAIQVLNQDKWTRETVQWLLNGTCPQFVSGLLESGKSELKKQVKPKAWLSRGPSPGPGRLLLVCHVSGFYPKPVWVKWMRGEQEQQGTQPGDILPNADETWYLRATLDVVAGEAAGLSCRVKHSSLEGQDIVLYWGGSYT

SEQ ID NO: 5 - protein, 99 aa, Homo sapiens [sequence omitted]

SEQ ID NO: 6 - protein, 488 aa, Homo sapiens
MGSLQPLATLYLLGMLVASSLGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGGGGEVPQRLFPLRCLQISSFANSSWTRTDGLAWLGELQTHSWSNDSDTVRSLKPWSQGTFSDQQWETLQHIFRVYRSSFTRDVKEFAKMLRLSYPLELQVSAGCEVHPGNASNNFFHVAFQGKDILSFQGTSWEPTQEAPLWVNLAIQVLNQDKWTRETVQWLLNGTCPQFVSGLLESGKSELKKQVKPKAWLSRGPSPGPGRLLLVCHVSGFYPKPVWVKWMRGEQEQQGTQPGDILPNADETWYLRATLDVVAGEAAGLSCRVKHSSLEGQDIVLYWGGSYTSGSGSGSGSLGGIFEAMKMELRDHHHHHHNWSHPQFEKGGGGSGGGGSGGSSAWSHPQFEKC

SEQ ID NO: 7 - DNA, 1467 bp, Homo sapiens
atgggttctctgcaaccgctggccaccttgtacctgctgggtatgctggtcgctagcagcctcggaatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggcggagggtctggaggagggggatctggaggtggaggctccggaggaggtggatcaggaggaggtggagaagtcccgcaaaggcttttccccctccgctgcctccagatctcgtccttcgccaatagcagctggacgcgcaccgacggcttggcgtggctgggggagctgcagacgcacagctggagcaacgactcggacaccgtccgctctctgaagccttggtcccagggcacgttcagcgaccagcagtgggagacgctgcagcatatatttcgggtttatcgaagcagcttcaccagggacgtgaaggaattcgccaaaatgctacgcttatcctatcccttggagctccaggtgtccgctggctgtgaggtgcaccctgggaacgcctcaaataacttcttccatgtagcatttcaaggaaaagatatcctgagtttccaaggaacttcttgggagccaacccaagaggccccactttgggtaaacttggccattcaagtgctcaaccaggacaagtggacgagggaaacagtgcagtggctccttaatggcacctgcccccaatttgtcagtggcctccttgagtcagggaagtcggaactgaagaagcaagtgaagcccaaggcctggctgtcccgtggccccagtcctggccctggccgtctgctgctggtgtgccatgtctcaggattctacccaaagcctgtatgggtgaagtggatgcggggtgagcaggagcagcagggcactcagccaggggacatcctgcccaatgctgacgagacatggtatctccgagcaaccctggatgtggtggctggggaggcagctggcctgtcctgtcgggtgaagcacagcagtctagagggccaggacatcgtcctctactggggtgggagctacacctccggtagtggtagtggtagtggatctctgggtggtatcttcgaggctatgaagatggagctgcgcgatcatcaccatcaccatcacaactggagccaccctcagttcgagaagggaggtggaggctcaggaggtggaggctctggaggctctagtgcatggagccaccctcagttcgagaagtgttga